(19) United States
(12) Patent Application Publication
(10) Pub. No.: US 2013/0006080 A1
(43) Pub. Date: Jan. 3, 2013

(54) LIVING BODY COMPONENT ANALYZING METHOD AND LIVING BODY COMPONENT ANALYZING APPARATUS

(75) Inventors: Seiki OKADA, Kobe (JP); Yoshihiro ASAKURA, Kobe (JP); Toshiyuki SATO, Kobe (JP); Kei HAGINO, Kobe (JP); Junko KOJIMA, Kobe (JP)

(73) Assignee: PANASONIC CORPORATION, Osaka (JP)

(21) Appl. No.: 13/614,616

(22) Filed: Sep. 13, 2012

Related U.S. Application Data
(63) Continuation of application No. PCT/JP2011/057558, filed on Mar. 28, 2011.

(30) Foreign Application Priority Data
Mar. 29, 2010 (JP) 2010-075807
Sep. 28, 2010 (JP) 2010-217638

(51) Int. Cl. A61B 5/00

(52) U.S. Cl. 600/362

(57) ABSTRACT

A living body component analyzing method that can accurately analyze a measurement target component even when the subject perspires is provided. A living body component analyzing method for analyzing a component contained in a tissue fluid extracted from the skin of a subject includes: a step of subjecting part of the skin of the subject to a process of facilitating extraction of the tissue fluid; a step of collecting a measurement target component from the skin subjected to the facilitation process; a step of collecting a first auxiliary component from the skin subjected to the facilitation process; a step of collecting a second auxiliary component contained in perspiration from the skin excluding the part subjected to the facilitation process; and a step of analyzing the measurement target component based on the collected measurement target component, the first auxiliary component and the second auxiliary component.

### RELATED APPLICATIONS

This application is a continuation of PCT/JP2011/057558 filed on Mar. 28, 2011, which claims priority to Japanese Application Nos. 2010-075807 filed on Mar. 29, 2010 and 2010-217638 filed on Sep. 28, 2010. The entire contents of these applications are incorporated herein by reference.

### TECHNICAL FIELD

The present invention relates to a living body component analyzing method and a living body component analyzing apparatus. More specifically, the present invention relates to method and apparatus for analyzing a measurement target component contained in tissue fluid extracted from the skin of a subject having undergone a process for facilitating the extraction of the tissue fluid.

### BACKGROUND ART

Conventionally, methods for measuring a measurement target component contained in tissue fluid extracted from the skin of a subject are known (e.g., see Patent Literature 1).

Patent Literature 1 discloses a method for calculating (estimating) the area under the blood glucose-time curve of a subject using the tissue fluid extracted from the skin of the subject. The method includes: forming micropores at the skin of a subject using a puncture device; applying a tissue fluid collecting sheet having a collecting material made of gel to the skin where the micropores are formed for a prescribed time (e.g., a time of 60 minutes or more), to thereby collect tissue fluid oozing from the skin. Then, the glucose amount and the sodium ion amount contained in the tissue fluid collected by the collecting material are measured. Based on the obtained glucose amount and sodium ion amount, the area under the blood glucose-time curve of the subject is estimated.

### CITATION LIST

### Patent Literature

Patent Literature 1: WO 2010/013808 A

### SUMMARY OF THE INVENTION

The method disclosed by Patent Literature 1 is based on the premise that the subject does not perspire. However, actually, there are some subjects who perspire during collection of the tissue fluid.

The present invention is made under such circumstances, and an object of the present invention is to provide a living body component analyzing method and a living body component analyzing apparatus that can accurately analyze a measurement target component obtained from a perspiring subject.

A living body component analyzing method of the present invention is a living body component analyzing method for analyzing a component contained in a tissue fluid extracted from a skin of a subject, including:

a step of subjecting part of a skin of a subject to a process of facilitating extraction of a tissue fluid;

a step of collecting a measurement target component from the skin subjected to the facilitation process;

a step of collecting a first auxiliary component from the skin subjected to the facilitation process;

a step of collecting a second auxiliary component contained in perspiration from the skin excluding the part of the skin subjected to the facilitation process; and

a step of analyzing the measurement target component based on the collected measurement target component, the collected first auxiliary component, and the collected second auxiliary component.

With the living body component analyzing method of the present invention, from the surface of the skin subjected to the facilitation process, the first auxiliary component attributed to the tissue fluid and perspiration is collected. On the other hand, at the surface of the skin not subjected to the facilitation process, the tissue fluid scarcely oozes out. Therefore, the second auxiliary component attributed solely to perspiration is collected. Accordingly, by collecting such a first auxiliary component and a second auxiliary component and comparing them with each other, it becomes possible to grasp to what extent the auxiliary component attributed to perspiration is mixed in the first auxiliary component. Thus, even when the subject perspires, according to the living body component analyzing method of the present invention, an accurate analysis result of the measurement target component can be generated based on the collected measurement target component, first auxiliary component, and second auxiliary component.

Preferably, the first auxiliary component and the second auxiliary component are collected in an identical period.

Preferably, the first auxiliary component and the second auxiliary component are collected at an identical arm.

The step of analyzing may include:

a first measurement step of measuring the collected second auxiliary component to acquire a first measurement value;

a step of comparing the first measurement value with a prescribed threshold value;

a second measurement step of measuring the collected measurement target component to acquire a second measurement value, when the first measurement value is smaller than the prescribed threshold value;

a third measurement step of measuring the collected first auxiliary component to acquire a third measurement value, when the first measurement value is smaller than the prescribed threshold value; and

a step of generating an analysis result including a value relating to an amount of the measurement target component based on the second and third measurement values.

The step of analyzing may include:

a first measurement step of measuring the collected second auxiliary component to acquire a first measurement value;

a second measurement step of measuring the collected measurement target component to acquire a second measurement value;

a third measurement step of measuring the collected first auxiliary component to acquire a third measurement value; and

a step of generating an analysis result of the measurement target component based on the first to third measurement values.

The step of generating the analysis result may include:

a step of comparing the first measurement value with the prescribed threshold value; and

a step of generating an analysis result including a value relating to an amount of the measurement target component based on the second and third measurement values and information indicative of the value having low reliability, when the first measurement value is equal to or greater than the prescribed threshold value.

The step of generating the analysis result may include:

a step of comparing the first measurement value with the prescribed threshold value; and

a step of generating an analysis result including a message expressing that the value relating to the amount of the measurement target component is not to be output, when the first measurement value is equal to or greater than the prescribed threshold value.

The step of generating the analysis result may include:

a step of comparing the first measurement value with the first threshold value and with a second threshold value, the second threshold value being greater than the first threshold value;

a step of generating an analysis result including a value relating to an amount of the measurement target component based on the second and third measurement values and information indicative of the value having low reliability, when the first measurement value is equal to or greater than the first threshold value and smaller than the second threshold value; and

a step of generating an analysis result including a message expressing that the value relating to the amount of the measurement target component is not to be output, when the first measurement value is equal to or greater than the second threshold value.

The first measurement value may be a value relating to an amount of the second auxiliary component;

the second measurement value may be a value relating to an amount of the measurement target component; and

the third measurement value may be a value relating to an amount of the first auxiliary component.

The step of analyzing may be a step of generating an analysis result of the measurement target component by correcting the second measurement value by a correction value obtained based on the first measurement value and the third measurement value.

The correction value may be a value obtained by subtracting the first measurement value from the third measurement value.

Each of the values relating to the amount may be an extraction amount of each of the auxiliary components per unit time.

The step of generating the analysis result may include:

a step of comparing the first measurement value with the first threshold value and with a second threshold value, the second threshold value being greater than the first threshold value;

a step of generating an analysis result of the measurement target component by correcting the second measurement value by a correction value based on the first and third measurement values, when the first measurement value is equal to or greater than the first threshold value and smaller than the second threshold value; and

a step of generating an analysis result including a message expressing that the value relating to the amount of the measurement target component is not to be output, when the first measurement value is equal to or greater than the second threshold value.

The measurement target component may be glucose.

The first auxiliary component and the second auxiliary component may be inorganic ions.

The first auxiliary component and the second auxiliary component may be of an identical type.

The inorganic ions may be sodium ions.

The measurement target component and the first auxiliary component can be collected by a collecting material disposed at an application face of a retainer sheet, the application face being capable of being applied to the skin of the subject.

Preferably, the collecting material is made of gel.

The living body component analyzing apparatus of the present invention is a living body component analyzing apparatus for analyzing a component contained in a tissue fluid extracted from a skin of a subject, including:

an acquiring unit that acquires information relating to a measurement target component and a first auxiliary component from a collection member having been disposed for a prescribed time at part of the skin of the subject, the part being subjected to a process of facilitating extraction of the component; and

an analyzing unit that analyzes the measurement target component based on the information relating to the measurement target component and to the first auxiliary component acquired by the acquiring unit, and based on information relating to a second auxiliary component contained in perspiration from the skin excluding the part of the skin subjected to the facilitation process.

Preferably, the living body component analyzing apparatus further includes a second acquiring unit that acquires information relating to the second auxiliary component.

Preferably, the living body component analyzing apparatus further includes an information receiving unit that receives the information relating to the second auxiliary component.

With the living body component analyzing method and the living body component analyzing apparatus of the present invention, the measurement target component of a perspiring subject can accurately be analyzed.

### BRIEF DESCRIPTION OF THE DRAWINGS

FIG. 1 is a perspective explanatory view showing the appearance of one embodiment of a living body component analyzing apparatus of the present invention;

FIG. 2 is a block diagram of the living body component analyzing apparatus shown in FIG. 1;

FIG. 3 is a schematic cross-sectional view showing the structure of a main-measurement purpose cartridge;

FIG. 4 is a perspective explanatory view of one example of a micropore forming apparatus that forms micropores at the skin of a subject;

FIG. 5 is a perspective view of a microneedle chip that is attached to the micropore forming apparatus shown in FIG. 4;

FIG. 6 is a cross-sectional explanatory view of skin at which micropores are formed by the micropore forming apparatus;

FIG. 7 is a perspective explanatory view of one example of a main-measurement purpose collection member;

FIG. 8 is a cross sectional view taken along the line A-A shown in FIG. 7;

FIG. 9 is a perspective explanatory view of one example of a perspiration-check purpose collection member;

FIG. 10 is a diagram describing the principle of measuring the conductivity of gel containing a second auxiliary component;

FIG. 11 is a diagram describing the principle of measuring the sodium ion concentration in gel containing the second auxiliary component;

FIG. 12 is a flowchart of a living body component analyzing method according to a first embodiment;

FIG. 13 is a graph showing the correlation between the glucose permeability and the extraction rate of sodium ion;

FIG. 14 is a graph showing the relationship between the measurement value deviation rate and the extraction rate of sodium ion J_{Na2};

FIG. 15 is a graph showing the relationship between the measurement value deviation rate and the Na relative value;

FIG. 16 is a graph showing the correlation between the glucose permeability and the extraction rate of sodium ion before the error cases are excluded;

FIG. 17 is a graph showing the correlation between the estimated blood glucose AUC value and the sampled blood glucose AUC value before the error cases are excluded;

FIG. 18 is a graph showing the relationship between the measurement value deviation rate and the non-puncture site extraction rate of sodium ion before the error cases are excluded;

FIG. 19 is a graph showing the correlation between the glucose permeability and the extraction rate of sodium ion after the error cases are excluded;

FIG. 20 is a graph showing the correlation between the estimated blood glucose AUC value and the sampled blood glucose AUC value after the error cases are excluded;

FIG. 21 is a flowchart of a living body component analyzing method according to a second embodiment;

FIG. 22 is a flowchart showing processing of a control unit according to the second embodiment;

FIG. 23 is a flowchart showing processing of a control unit according to a third embodiment;

FIG. 24 is a perspective explanatory view of an integrated collection member;

FIG. 25 is a flowchart of a living body component analyzing method according to a fourth embodiment;

FIG. 26 is a flowchart showing the process of a control unit according to the fourth embodiment;

FIG. 27 is a graph showing the correlation between the glucose permeability and the extraction rate of sodium ion when the perspiration correction is not performed;

FIG. 28 is a graph showing the relationship between the extraction rate of sodium ion and the measurement value deviation rate at a non-puncture site;

FIG. 29 is a graph showing the correlation between the glucose permeability and the extraction rate of sodium ion when the perspiration correction is performed; and

FIG. 30 is a graph showing the measurement value deviation rate before and after the perspiration correction.

### DETAILED DESCRIPTION OF THE PREFERRED EMBODIMENTS

In the following, with reference to the accompanying drawings, a description will be given in detail of embodiments of a living body component analyzing method and a living body component analyzing apparatus of the present invention.

### First Embodiment

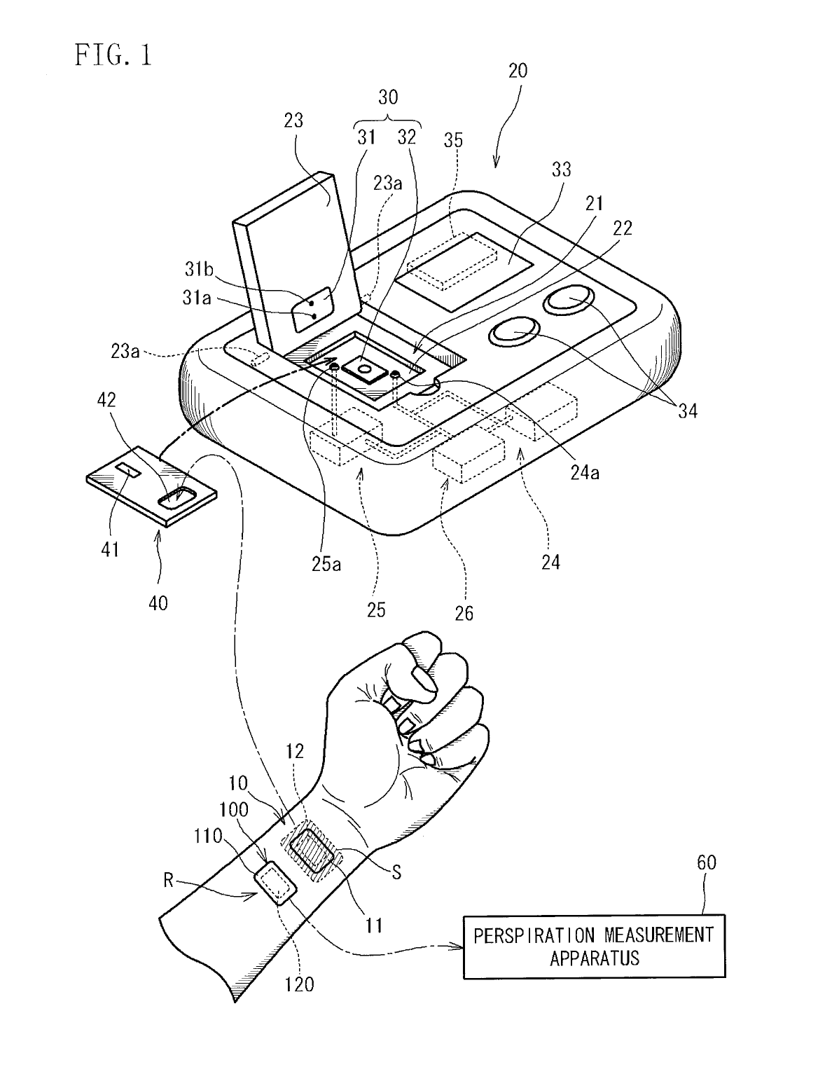

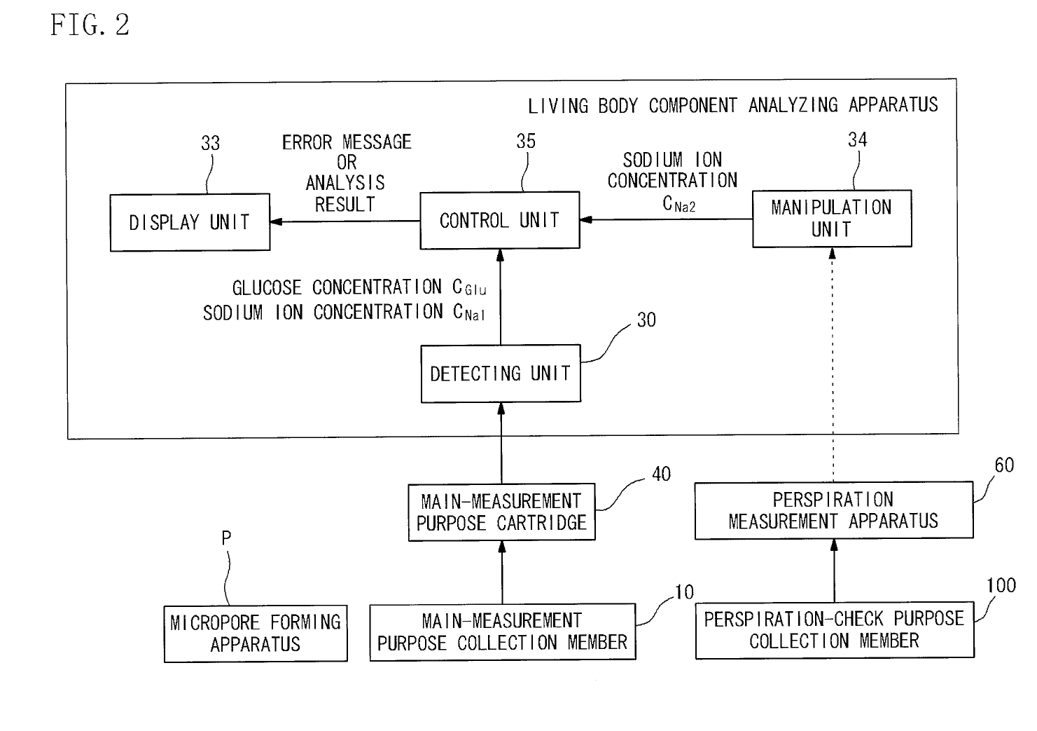

FIG. 1 is a perspective explanatory view showing the appearance of a living body component analyzing apparatus 20 according to one embodiment of the present invention. FIG. 2 is a block diagram of the living body component analyzing apparatus shown in FIG. 1. First, with reference to FIG. 1, an overview of a living body component analyzing method will be described.

As will be described later, the living body component analyzing method according to the present embodiment is a method including: forming micropores at the skin of a subject; extracting tissue fluid via the micropores; collecting glucose and sodium ions contained in the extracted tissue fluid; and analyzing blood-glucose (blood sugar) of the subject based on the concentration of the collected glucose and sodium ions. More specifically, it is a method of calculating the area under the blood glucose-time curve (blood glucose AUC).

When the subject perspires, sodium ions attributed to perspiration are collected as being superimposed on the sodium ions attributed to tissue fluid, and the sodium ion concentration becomes high. With the living body component analyzing method according to the present embodiment, the area under the blood glucose-time curve of the subject is estimated based on the sodium ions collected with the glucose. Therefore, when the sodium ions attributed to perspiration are excessively collected, the reliability of calculated blood glucose AUC may be reduced.

Accordingly, in the method according to the present embodiment, a main-measurement purpose collection member 10 is applied to skin S where micropores are formed, and a perspiration-check purpose collection member 100 is applied to skin R where no micropores are formed. In this state, the main-measurement purpose collection member 10 is allowed to collect glucose and sodium ions contained in tissue fluid. At the same time, the perspiration-check purpose collection member 100 is allowed to collect sodium ions contained in perspiration from the skin where no micropores are formed. Then, the sodium ions collected by the perspiration-check purpose collection member 100 are measured (hereinafter also referred to as the perspiration measurement). When the sodium ion concentration is higher than a threshold value, an error message that prompts not to perform measurement of the concentrations of glucose and sodium ions collected by the main-measurement purpose collection member 10 (hereinafter also referred to as the main measurement), and analysis of the blood glucose AUC based on the concentrations as well. This prevents the analysis result of the blood glucose AUC with low reliability from being output.

### [Living Body Component Analyzing Apparatus]

The living body component analyzing apparatus 20 is an apparatus that performs measurement of glucose and sodium ions contained in tissue fluid collected in the collecting material 12 of the main-measurement purpose collection member 10 whose description will follow (hereinafter also referred to as the main measurement); acquires a glucose concentration (C_{Glu}) and a sodium ion concentration (C_{Na1}); calculates the area under the blood glucose-time curve (hereinafter also referred to as the blood glucose AUC) of the subject based on the acquired C_{Glu} and C_{Na1}; and generates and displays an analysis result including the blood glucose AUC. The living body component analyzing apparatus 20 comprises a detecting unit 30, a control unit 35 that includes an analyzing unit, a display unit 33 that displays an analysis result or an error message, and a manipulation button 34 as a manipulation unit that instructs start of measurement.

The living body component analyzing apparatus 20 includes a thick rectangular parallelepiped-shaped housing. At the top plate on the top face of the housing, a recessed portion 21 is formed. The recessed portion 21 is provided with a cartridge disposition portion 22 being a recessed portion whose depth is greater than the recessed portion 21. Further, a movable top plate 23 is coupled to the recessed portion 21. The thickness of the movable top plate 23 is substantially as great as the height of the sidewall of the recessed portion 21. The movable top plate 23 can be stored in the recessed portion 21 by being folded about a support shaft 23a from the state shown in FIG. 1, or can stand up as shown in FIG. 1 from the state being stored in the recessed portion 21. The cartridge disposition portion 22 is large enough to store a main-measurement purpose cartridge 40 whose description will follow.

The movable top plate 23 is supported by the support shaft 23a so as to be biased in the direction to be stored in the recessed portion 21. Accordingly, the main-measurement purpose cartridge 40 disposed in the cartridge disposition portion 22 is pressed from above by the movable top plate 23.

The detecting unit 30 acquires information of the component contained in the tissue fluid collected by the collecting material 12 of the main-measurement purpose collection member 10, and includes a glucose detecting unit 31 that detects a glucose concentration C_{Glu} being a measurement target component, and a sodium ion detecting unit 32 that detects a sodium ions concentration C_{Na1} being a first auxiliary component.

The glucose detecting unit 31 is provided at the back face of the movable top plate 23. That is, it is provided on the face that opposes to the cartridge disposition portion 22 when the movable top plate 23 is stored in the recessed portion 21. The glucose detecting unit 31 comprises a light source 31a for emitting light, and a light receiving portion 31b for receiving the reflected light of the light emitted by the light source 31a. Thus, the glucose detecting unit 31 is so structured as to be capable of emitting light to the light to the main-measurement purpose cartridge 40 disposed in the cartridge disposition portion 22 and to receive the reflected light from the main-measurement purpose cartridge 40 to which the light has been emitted.

The sodium ion detecting unit 32 is provided at the bottom face of the cartridge positioning portion 22. The sodium ion detecting unit 32 is provided with a rectangular plate-like member provided at the bottom face of the cartridge disposition portion 22, and a pair of sodium ion concentration measurement-purpose electrodes is provided at the substantially central portion of the plate-like member. The sodium ion concentration measurement-purpose electrode includes a sodium ion selective electrode made of a silver/silver chloride provided with a sodium ion selective membrane, and a silver/silver chloride electrode being the counter electrode.

The control unit 35 is provided inside the living body component analyzing apparatus 20, and includes a CPU being an analyzing unit, and ROM and RAM each being a storage unit. The CPU reads and executes the program stored in the ROM, to control the operations of each unit. The RAM is used as the expansion area of the program when the program stored in the ROM is executed.

The living body component analyzing apparatus 20 includes therein: a supply unit 24 made up of a pump, a tank 26 that stores recovery liquid being pure water for recovering the tissue fluid collected by the collecting material 12 of the main-measurement purpose collection member 10; and a waste fluid tank 25 that stores waste fluid. The supply unit 24 sends air to the tank 26, to thereby inject the recovery liquid stored in the tank 26 to the main-measurement purpose cartridge 40 disposed in the cartridge disposition portion 22 via a nipple 24a.

The waste fluid tank 25 is a mechanism to which the pure water delivered by the supply unit 24 to the main-measurement purpose cartridge 40 is discharged. The waste fluid tank 25 stores the discharged liquid via a nipple 25a.

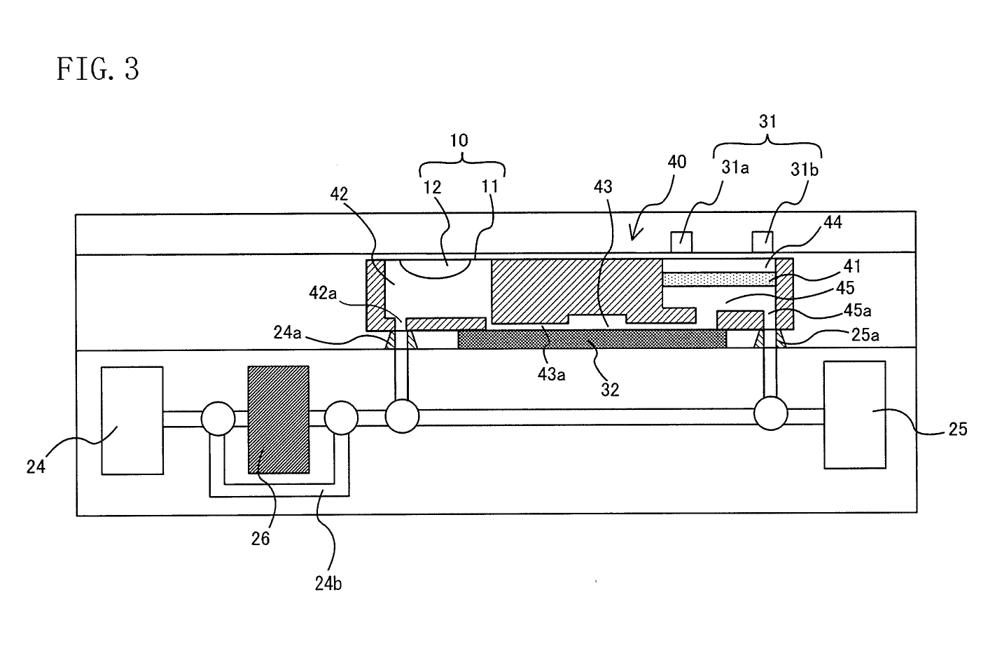

FIG. 3 is a schematic cross-sectional view showing the state where the main-measurement purpose cartridge 40 is disposed in the cartridge disposition portion 22. First, with reference to FIG. 3, a description will be given of the structure of the main-measurement purpose cartridge 40.

The main-measurement purpose cartridge 40 comprises, as its main constituents, a gel storage unit 42, a glucose reactant 41, and an optical waveguide member 44. The gel storage unit 42 is formed by a recessed portion formed on the surface of the main-measurement purpose cartridge 40. At the bottom portion of the gel storage unit 42, an injection port 42a that communicates with the nipple 24a provided at the cartridge disposition portion 22 is provided. At the bottom face of the main-measurement purpose cartridge 40, a groove that communicates with the gel storage unit 42 is formed. A flow channel 43a is formed by the groove and the sodium ion detecting unit 32 provided at the bottom portion of the cartridge disposition portion 22. Part of the flow channel 43a is a first reservoir 43 in which sodium ion concentration is detected by the sodium ion detecting unit 32. The downstream of the flow channel 43a communicates with the second reservoir 45. The second reservoir 45 is formed by a recessed portion provided at the front face of the main-measurement purpose cartridge 40. The opening of the second reservoir is closed by the optical waveguide member 44 having an optical waveguide. At the bottom face of the optical waveguide member 44, the glucose reactant 41 that discolors upon reaction with glucose is provided. Provided at the bottom portion of the second reservoir is a discharge port 45a that communicates with the nipple 25a provided at the cartridge disposition portion 22.

The living body component analyzing apparatus 20 measures glucose concentration C_{Glu} and sodium ions concentration C_{Na1} contained in the tissue fluid collected by the main-measurement purpose collection member 10 in the following manner. First, as represented by alternate long and short dash lines in FIG. 1, the main-measurement purpose collection member 10 having been applied to the skin S of the subject for a prescribed time is removed from the skin, and is applied to the gel storage unit 42 of the main-measurement purpose cartridge 40. The main-measurement purpose cartridge 40 is disposed in the cartridge disposition portion 22 of the living body component analyzing apparatus 20, and the movable top plate 23 is closed.

When start of measurement is instructed by the manipulation button 34, air is supplied from the supply unit 24 to the tank 26, and recovery liquid is sent from the tank 26 to the nipple 24a. The recovery liquid is injected from the injection port 42a into the gel storage unit 42, and the gel storage unit 42 is filled with the recovery liquid. In this state, when a prescribed time has elapsed, the tissue fluid collected by the collecting material 12 diffuses into the recovery liquid. When the prescribed time has elapsed, the supply unit 24 supplies air into the gel storage unit 42 through a bypass route 24b. Thus, the liquid in the gel storage unit 42 is sent to the first reservoir 43 and the second reservoir 45 via the flow channel 43a.

The sodium ion detecting unit 32 acquires the current value by applying a constant voltage through the sodium ion concentration measurement-purpose electrode to the liquid accumulated in the first reservoir 43. The current value at this time is proportional to the concentration of sodium ions contained in the liquid. The sodium ion detecting unit 32 outputs the acquired current value to the control unit 35 as a detection signal. The control unit 35 acquires the sodium ion concentration C_{Na1} based on the current value included in the detection signal and the calibration curve previously stored in the storage unit of the control unit 35.

In the second reservoir, the glucose in the recovery liquid and the glucose reactant 41 react with each other, and the glucose reactant 41 discolors. The glucose detecting unit 31 emits light from the light source 31a toward the optical waveguide member 44, and the light output from the optical waveguide member 44 is received by the light receiving portion 31b. When the light is emitted from the light source 31a, while the light is absorbed by the discolored glucose reactant 41, the light repeatedly reflects within the optical waveguide member 44, and enters the light receiving portion 31b. The received light amount in the light receiving portion 31b is proportional to the discoloring degree of the glucose reactant 41, and this discoloring degree is proportional to the glucose amount in the recovery liquid. The glucose detecting unit 31 outputs the acquired received light amount to the control unit 35 as a detection signal. The control unit 35 acquires a glucose concentration C_{Glu} based on the received light amount included in the detection signal and the calibration curve previously stored in the storage unit of the control unit 35.

When the sodium ion concentration C_{Na1} and the glucose concentration C_{Glu} are acquired, the supply unit 24 further sends air to the main-measurement purpose cartridge 40. Thus, the recovery liquid is sent to the waste fluid tank 25 via the discharge port 45a and the nipple 25a, and a sequence of measurement steps ends.

### [Micropore Forming Apparatus]

Next, a description will be given of one example of a micropore forming apparatus that forms micropores at the skin of a subject. The micropore forming apparatus is an apparatus that forms a multitude of micropores at part of the skin of a subject, so as to facilitate extraction of tissue fluid from the skin of the subject. In the present embodiment, glucose and sodium ions are collected from the skin S (see FIG. 1) of the subject where micropores for tissue fluid extraction facilitation are formed. At the same time, as will be described later, sodium ions contained in perspiration from the skin R of the subject where no micropores are formed are collected.

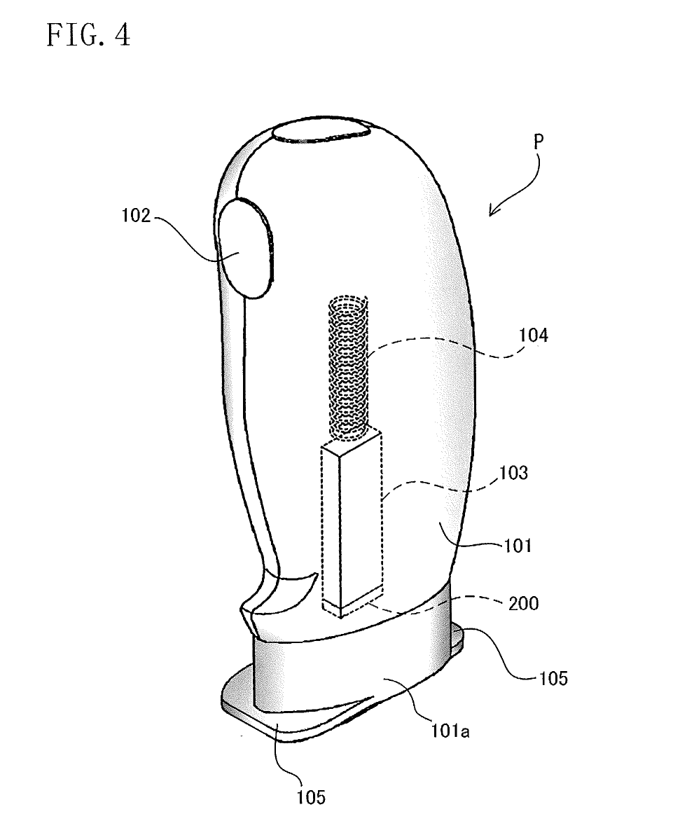

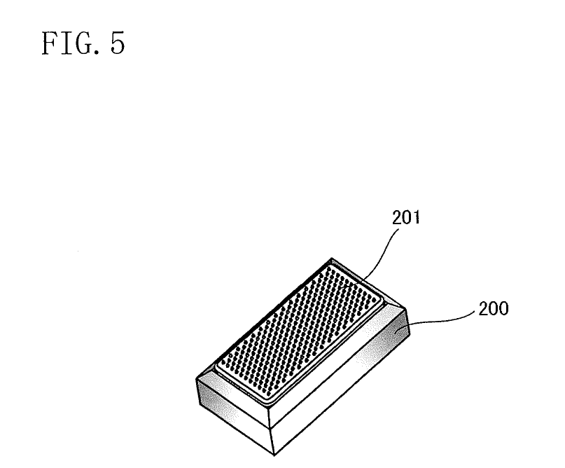

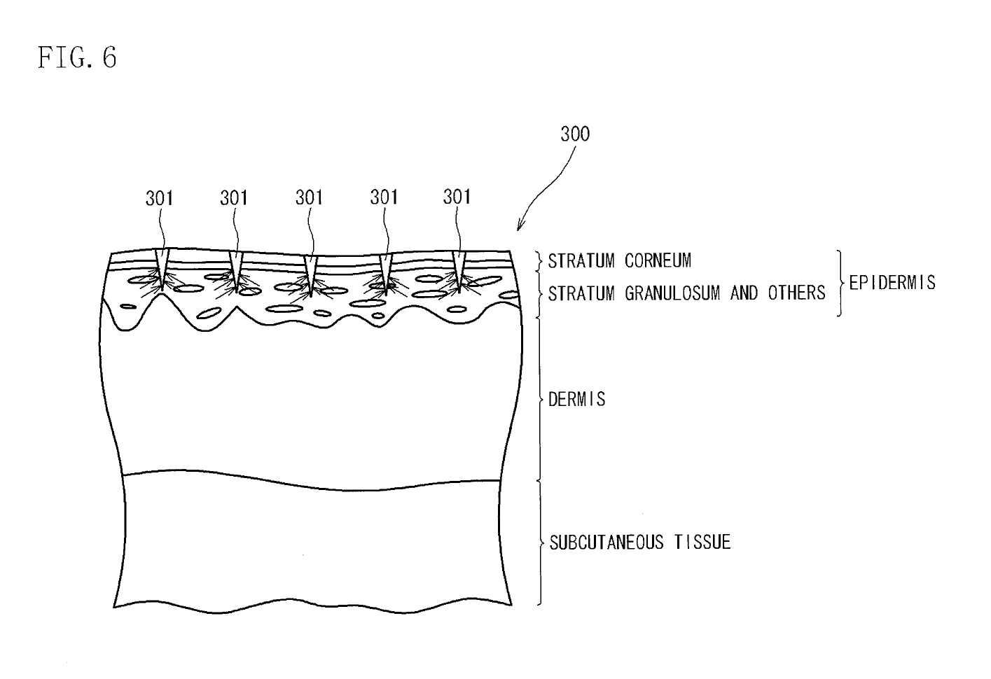

FIG. 4 is a perspective explanatory view of a puncture device P according to one example of the micropore forming apparatus which is used to form micropores for tissue fluid extraction facilitation at the skin of the subject in the living body component analyzing method of the present invention. FIG. 5 is a perspective view of a microneedle chip 200 attached to the puncture device P shown in FIG. 4. FIG. 6 is a cross-sectional explanatory view of the skin S where the micropores are formed by the puncture device P.

As shown in FIGS. 4 to 6, the puncture device P is an apparatus that forms extraction pores (micropores 301) for tissue fluid at the skin 300 of a subject in the following manner. The sterilized microneedle chip 200 is attached to the puncture device P, and microneedles 201 of the microneedle chip 200 are allowed to abut on the epidermis (the skin 300 of the subject) of the living body. The microneedles 201 of the microneedle chip 200 is designed to have dimension such that when the micropores 301 are formed by the puncture device P, the micropores 301 stay within the epidermis of the skin 300, and do not reach the dermis.

As shown in FIG. 4, the puncture device P comprises a housing 101, a release button 102 provided at the surface of the housing 101, and an array chuck 103 and a spring member 104 provided inside the housing 101. At the bottom end face (the face that abuts on the skin) of the bottom portion 101a of the housing 101, an opening (not shown) through which the microneedle chip 200 can pass is formed. The spring member 104 has a function of biasing the array chuck 103 in the puncture direction. To the bottom end of the array chuck 103, the microneedle chip 200 can be attached. At the bottom face of the microneedle chip 200, a plurality of microneedles 201 are formed. The bottom face of the microneedle chip 200 is as large as 10 mm (long side) 5 mm (short side). Further, the puncture device P has a fixing mechanism (not shown) that fixes the array chuck 103 in the state where the array chuck 103 is pushed upward (in the counter-puncture direction) against the biasing force of the spring member 104. When the user (subject) presses down the release button 102, the fixation of the array chuck 103 by the fixing mechanism is released, and the array chuck 103 shifts in the puncture direction by the biasing force of the spring member 104. This allows the microneedles 201 of the microneedle chip 200 projecting from the opening to puncture the skin. It is to be noted that, in FIG. 4, numeral 105 is a convex portion formed at the bottom portion 101a of the housing 101. When the puncture device P is used, the back face of the convex portion 105 is allowed to abut on a prescribed part of the skin of the subject.

### [Main-Measurement Purpose Collection Member]

Next, a description will be given of the main-measurement purpose collection member 10 for collecting the tissue fluid from the skin of a subject. The main-measurement purpose collection member 10 is applied to the skin of a subject for collecting the tissue fluid from the skin of the subject, and peeled off from the skin after a lapse of a prescribed time.

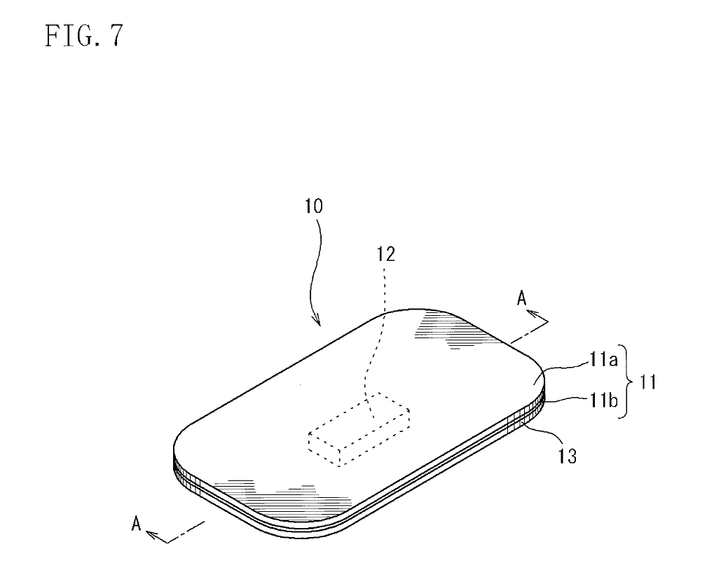

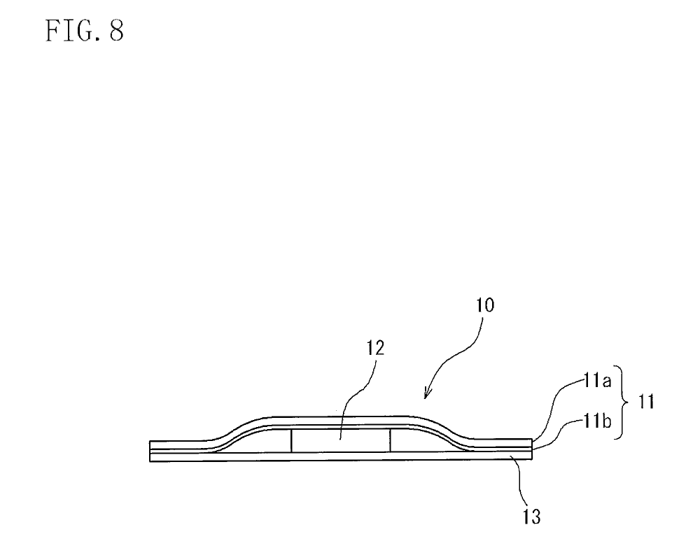

FIG. 7 is a perspective explanatory view of the main-measurement purpose collection member 10 comprising the retainer sheet 11 and the collecting material 12 retained by the retainer sheet 11. FIG. 8 is a cross sectional view taken along the line A-A shown in FIG. 7.

The collecting material 12 is made of water-retentive gel that can retain the tissue fluid extracted from the skin of a subject, and retains pure water as an extraction medium. The gel is not particularly limited so long as it can collect the tissue fluid. Preferably, it is the gel formed by at least one type of hydrophilic polymer selected from the group consisting of polyvinyl alcohol and polyvinylpyrrolidone. The hydrophilic polymer forming the gel may be solely polyvinyl alcohol or solely polyvinylpyrrolidone, or may be the mixture of them. More preferably, it is solely polyvinyl alcohol or the mixture of polyvinyl alcohol and polyvinylpyrrolidone.

The gel can be formed by a method of cross-linking hydrophilic polymer in an aqueous solution. The gel is formed by a method of cross-linking hydrophilic polymer, including: applying a hydrophilic polymer aqueous solution on a base material to form a coat; and cross-linking hydrophilic polymer contained in the coat. Cross-linking method of hydrophilic polymer includes the chemical cross-linking method, the radiation cross-linking method and the like. It is desirable to employ the radiation cross-linking method, because various chemical substances are less likely to be mixed in the gel as impurities.

The collecting material 12 has a rectangular parallelepiped-shape in the example shown in FIGS. 7 to 8, and the size of the face being brought into contact with the skin is as large as 7 mm 12 mm. It is to be noted that, the shape and size of the collecting material 12 is not limited thereto.

The retainer sheet 11 is structured with an oval-shaped sheet body 11a, and an adhesive agent layer 11b formed on one face of the sheet body 11a. The face where the adhesive agent layer 11b is formed is the adhesive face. The collecting material 12 is arranged at the substantially central portion of the release sheet 13 that similarly functions as an oval-shaped mounting. The retainer sheet 11 is bonded to the release sheet 13 so as to cover the collecting material 12. The collecting material 12 is retained in the retainer sheet 11 by part of the adhesive face of the retainer sheet 11. The area of the retainer sheet 11 is large enough to cover the collecting material 12, so as to prevent the collecting material 12 from being dried when the tissue fluid is collected. That is, by allowing the retainer sheet 11 to cover the collecting material 12, the interface between the skin and the retainer sheet 11 can be kept air-tight when the tissue fluid is collected. Thus, the moisture contained in the collecting material 12 can be suppressed from being vaporized when the tissue fluid is collected.

The sheet body 11a of the retainer sheet 11 is colorless and transparent or colored and transparent. Therefore, the collecting material 12 retained in the retainer sheet 11 can visually be checked from the front side of the sheet body 11a (the face opposite to the adhesive agent layer 11b). The sheet body 11a is preferably of low moisture permeability for preventing vaporization of the tissue fluid or drying of the collecting material. Exemplary materials of the sheet body 11a may include a polyethylene film, a polypropylene film, a polyester film, a polyurethane film and the like. Among others, a polyethylene film and a polyester film are preferable. Though the thickness of the sheet body 11a is not particularly limited, it is approximately 0.025 to 0.5 mm.

The main-measurement purpose collection member 10 is applied to the skin 300 of the subject by the adhesive face of the retainer sheet 11, such that the collecting material 12 is disposed at the micropore formation region S of the subject (the region where a plurality of micropores 301 are formed at the skin 300 of the subject by the puncture device P so as to facilitate extraction of the tissue fluid). Then, by leaving the collecting material 12 in the state where the collecting material 12 is disposed at the micropore formation region for a prescribed time, for example 60 minutes or more, preferably 180 minutes or more, the components contained in the tissue fluid extracted through the micropores are collected by the collecting material 12.

### [Perspiration-Check Purpose Collection Member]

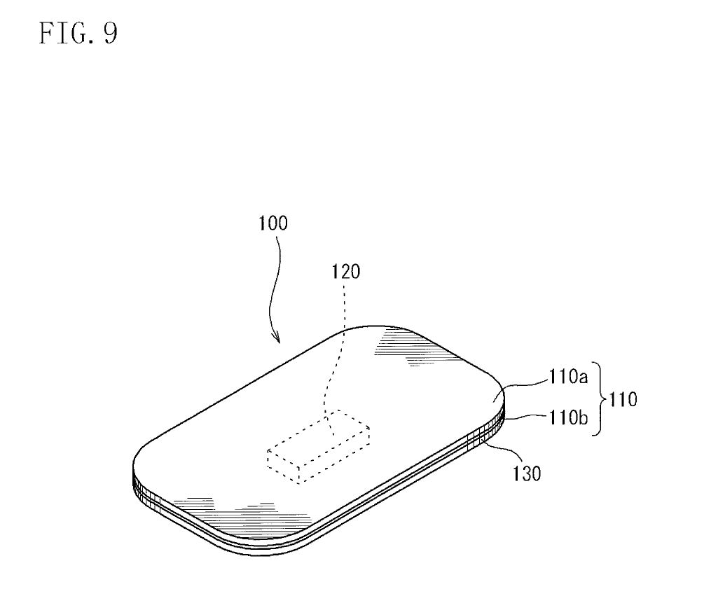

Next, a description will be given of the perspiration-check purpose collection member 100 that collects perspiration from the skin of a subject. FIG. 9 is a perspective view showing the structure of the perspiration-check purpose collection member 100 according to the present embodiment. The perspiration-check purpose collection member 100 has the same structure as the main-measurement purpose collection member 10 as described above, and comprises a retainer sheet 110, a collecting material 120 retained by the retainer sheet 110, and a release sheet 130. The structure of each constituent of the perspiration-check purpose collection member 100 is the same as that of the main-measurement purpose collection member 10 shown in FIGS. 7 and 8, and therefore a detailed description thereof will not be repeated.

### [Perspiration Measurement Apparatus]

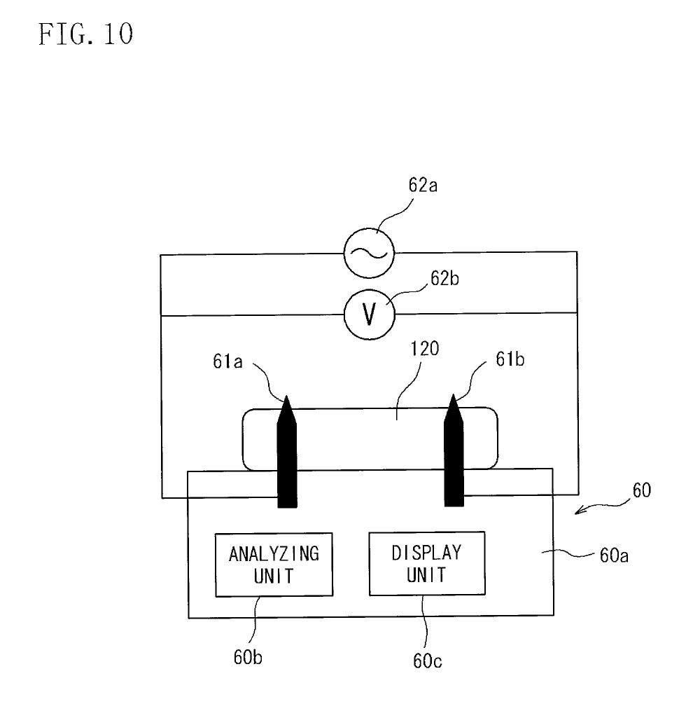

FIG. 10 is a schematic explanatory view of a perspiration measurement apparatus used in the living body component analyzing method according to the present embodiment. The perspiration measurement apparatus 60 comprises a pedestal 60a on which the collecting material 120 of the perspiration-check purpose collection member 100 is placed, opposing electrodes 61a and 61b provided on the top face of the pedestal 60a, an AC power supply 62a, a voltmeter 62b that measures the voltage between the opposing electrodes 61a and 61b, an analyzing unit 60b, and a display unit 60c. When the collecting material 120 is placed on the pedestal 60a, the opposing electrodes 61a and 61b are inserted into the collecting material 120, and the opposing electrodes 61a and 61b are short-circuited via the collecting material 120. In this state, when a voltage is applied by the AC power supply 62a, the voltage between the opposing electrodes 61a and 61b is measured by the voltmeter 62b. The analyzing unit 60b analyzes the concentration C_{Na2} of the sodium ions collected by the collecting material 12 of the perspiration-check purpose collection member 100 based on the measured voltage value and the calibration curve, and allows the display unit 60c to display the sodium ion concentration C_{Na2}.

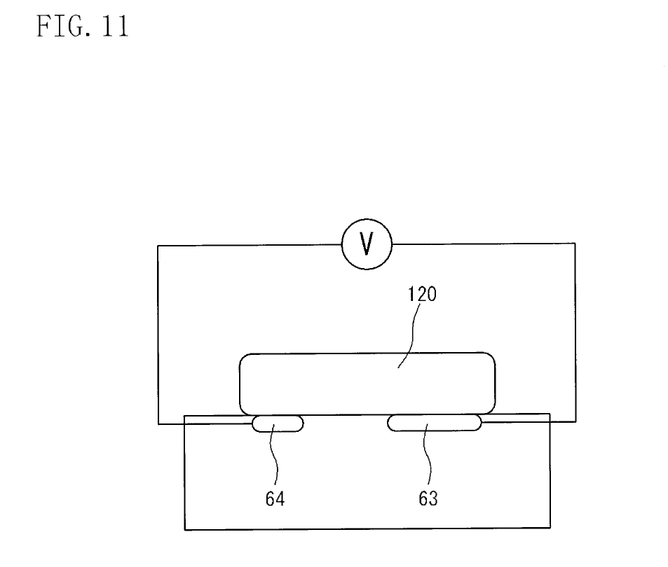

Further, as shown in FIG. 11, the perspiration measurement apparatus may include a pair of sodium ion concentration measurement-purpose electrodes comprising a sodium ion selective electrode 63 made of silver/silver chloride having a sodium ion selective membrane and a silver/silver chloride electrode 64 being the opposing electrode.

### [Living Body Component Analyzing Method]

Next, a description will be given of one embodiment of the living body component analyzing method according to the first embodiment of the present invention.

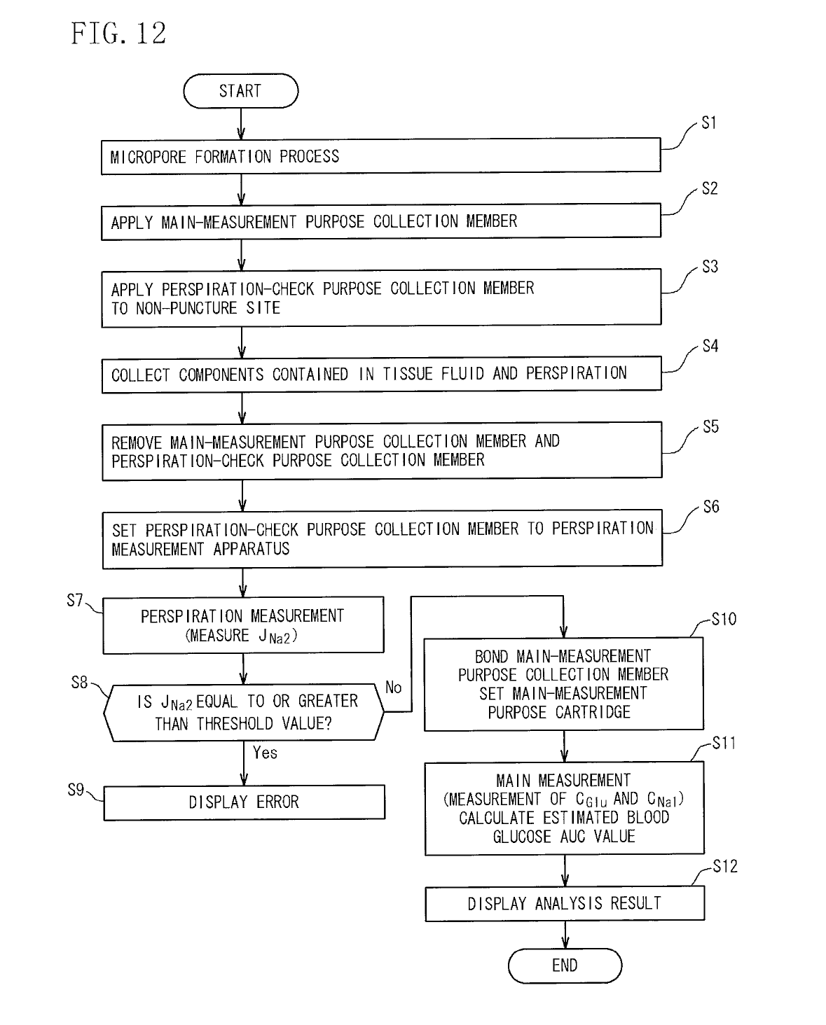

FIG. 12 is a flowchart of the living body component analyzing method according to the first embodiment.

First, in Step S1, micropores are formed at the skin of a subject using the puncture device shown in FIG. 4. Specifically, the skin 300 of the subject is cleaned using alcohol or the like, to remove any substance that acts as the disturbance factor (e.g., dust) to the measurement result. Thereafter, to the skin of the subject, the convex portion 105 of the puncture device P to which the microneedle chip 200 is attached is disposed. Next, the release button 102 is pressed, to allow the microneedles 201 of the microneedle chip 200 to be brought into contact with the skin 300 of the subject. Thus, micropores 301 are formed at the skin 300. Formation of such micropores can facilitate extraction of tissue fluid from the skin 300.

Next, in Step S2, the puncture device P is separated from the skin 300 of the subject, and the retainer sheet 11 of the main-measurement purpose collection member 10 is applied to the skin 300 of the subject such that the collecting material 12 is disposed at the region S where micropores 301 are formed (the micropore formation region) (see FIG. 1).

Next, in Step S3, the perspiration-check purpose collection member 100 is applied to the non-puncture site R, e.g., the skin near the micropore formation region of the subject. The micropores are normally formed at the arm of the subject. Though it is possible to apply the main-measurement purpose collection member 10 and the perspiration-check purpose collection member 100 to separate arms, it is preferable that the main-measurement purpose collection member 10 and the perspiration-check purpose collection member 100 are applied to the same arm from the viewpoint of uniformizing the measurement condition as much as possible. By applying them to the same arm, even in the case where the perspiration amount is different between the right arm and the left arm, the difference in the collected sodium ions attributed to perspiration between the main-measurement purpose collection member 10 and the perspiration-check purpose collection member 100 can be reduced.

Next, in Step S4, the tissue fluid from the skin of the subject is extracted into the main-measurement purpose collection member 10, and glucose and sodium ions contained in the tissue fluid are collected and accumulated in the collecting material 12 of the main-measurement purpose collection member 10. At this time, in the case where the subject perspires, the tissue fluid and also the sodium ions included in the perspiration are collected from the skin of the subject into the main-measurement purpose collection member 10. At the same time, by the perspiration-check purpose collection member 100, the sodium ions contained in the perspiration are collected. The collection time is, for example, approximately 60 minutes to 180 minutes.

Next, in Step S5, the main-measurement purpose collection member 10 and the perspiration-check purpose collection member 100 are removed from the skin of the subject.

Steps S6 to S11 are the steps of analyzing the components collected in Step S4.

First, in Step S6, the perspiration-check purpose collection member 100 removed from the skin of the subject is set to the perspiration measurement apparatus 60. The perspiration-check purpose collection member 100 is set to the perspiration measurement apparatus 60, such that the opposing electrodes 61a and 61b of the perspiration measurement apparatus 60 are buried in the collecting material 120 of the perspiration-check purpose collection member 100.

Next, in Step S7, by measuring the conductivity of the collecting material 120 of the perspiration-check purpose collection member 100, the sodium ion concentration C_{Na2} contained in the collecting material 120 is measured. The sodium ion amount estimated from the conductivity of the gel has been confirmed to have high correlation with the sodium ion amount which is separately measured using the ion chromatography. Accordingly, by the relatively easy method of measuring the conductivity of the gel, the sodium ion amount in the gel can be estimated. The sodium ion concentration C_{Na2} measured by the perspiration measurement apparatus 60 is displayed on the display unit 60c.

Next, in Step S8, the sodium ion concentration C_{Na2} measured in Step S7 is input to the living body component analyzing apparatus 20 by the manipulation button 34. Next, in Step S8, the control unit 35 determines as to whether or not the input sodium ion concentration C_{Na2} is higher than a prescribed threshold value. When the control unit 35 determines that the sodium ion concentration C_{Na2} is higher than the threshold value, the control unit 35 displays an error message (to the effect that execution of the main measurement cannot guarantee the accuracy because of the great perspiration amount) on the display unit 33. The main measurement (measurement of the glucose concentration C_{Glu} and the sodium ion concentration C_{Na1}, and calculation of the estimated blood glucose AUC value) is performed using the main-measurement purpose cartridge 40 described above. Since the main-measurement purpose cartridge 40 is a disposable cartridge containing the glucose reactant 41, by checking the perspiration amount and prompting to stop the analysis whose accuracy is low, any wasteful consumption of the main-measurement purpose cartridge 40 can be suppressed. The threshold value can previously be obtained from the experimental data, whose description will follow, as to the estimated blood glucose AUC value, the sampled blood glucose AUC, and the perspiration amount in the following manner, for example.

### [Setting of Threshold Value]

As an index for classifying the case with great perspiration and the case with small perspiration, one of the following can be used: (1) a threshold value for the extraction rate of sodium ion at the non-puncture site (a total amount of sodium ions collected by the perspiration-check purpose collection member 100 per unit time); and (2) a relative value of the extraction rate of sodium ion at the non-puncture site (a total amount of sodium ions collected by the perspiration-check purpose collection member 100 per unit time) relative to the extraction rate of sodium ion obtained from the sodium ions concentration C_{Na1} collected by the main-measurement purpose collection member 10 (hereinafter referred to as “Na relative value”). By setting a threshold value for each of the indexes, and comparing the index obtained from the subject with the threshold value, it becomes possible to determine whether or not a highly reliable blood glucose AUC can be obtained, based on the glucose concentration C_{Glu} and the sodium ion concentration C_{Na1}. Such threshold values can be obtained by an experiment. In the following, an example of the threshold values that are empirically set is described.

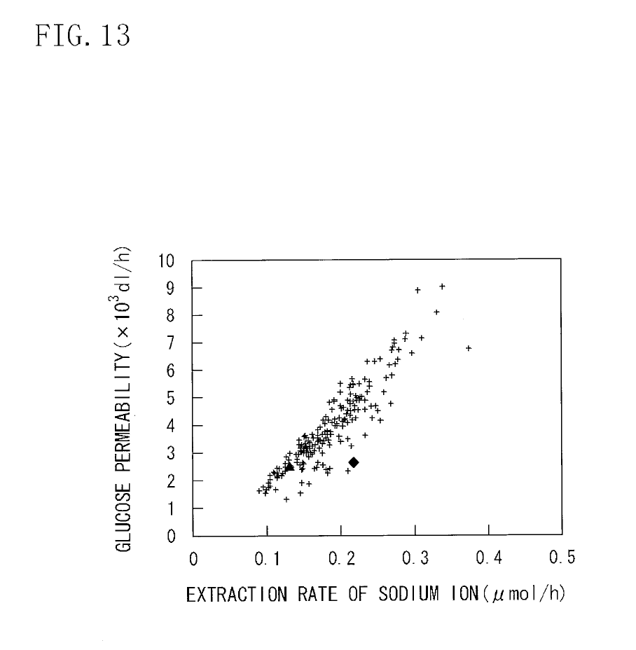

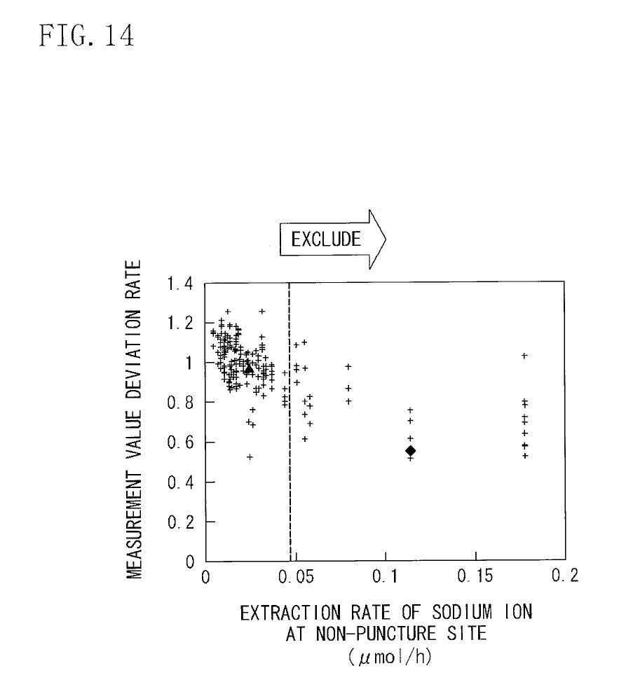

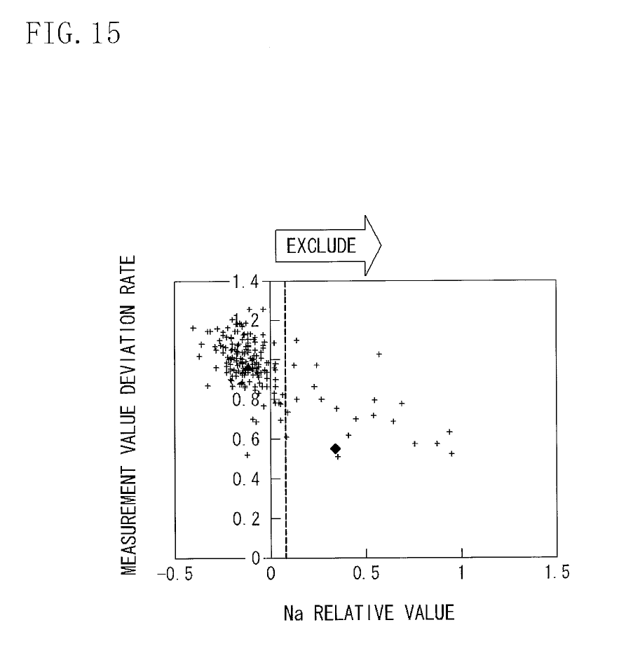

FIG. 13 is a graph showing the relationship between the glucose permeability (the vertical axis) and the extraction rate of sodium ion (the horizontal axis) relating to a plurality of cases. FIG. 14 is a graph in which data shown in FIG. 13 is divided based on the threshold value for the extraction rate of sodium ion J_{Na2}. FIG. 15 is a graph in which data shown in FIG. 13 is divided based on the threshold value for the relative Na value.

In acquiring the data shown in FIGS. 13 to 15, in parallel with the collection of tissue fluid and components included in perspiration according to the present embodiment, blood sampling was performed for a plurality of times every prescribed time period. Based on the glucose and sodium ions included in the tissue fluid and Formula (1) shown below, the blood glucose AUC was calculated (this is referred to as the estimated blood glucose AUC). Further, the blood glucose AUC was calculated by the known trapezoidal approximation based on the blood glucose values at a plurality of time points obtained by a plurality of times of blood sampling (this is referred to as the sampled blood glucose AUC).

The glucose permeability indicated by the vertical axis in FIG. 13 is the value which is the glucose amount collected by the main-measurement purpose collection member 10 being divided by the sampled blood glucose AUC. That is, the glucose permeability represents the ratio of the glucose amount extracted outside the body to the blood glucose AUC inside the body. On the other hand, the extraction rate of sodium ion is the amount of sodium ions collected by the main-measurement purpose collecting material 10 per unit time.

Out of the cases shown in FIG. 13, the example with great perspiration (symbol ♦) and the example with small perspiration (symbol ▴) are extracted. The respective measurement results are shown in the following Table 1.

TABLE 1

| | | JNa1 | JNa2 | Na relative |
|---|---|---|---|---|
| | Symbol | (μmol/h) | (μmol/h) | value |
| Example with | ♦ | 0.2175 | 0.01139 | 0.3076 |
| great | | | | |
| perspiration | | | | |
| Example with | ▴ | 0.1312 | 0.02436 | −0.1726 |
| small | | | | |
| perspiration | | | | |

It is to be noted that the Na relative value was obtained by the following formula.

Na relative value={(J_{Na2})□(constant γ)}÷(J_{Na1})

Here, calculation was performed with the constant γ=0.047. The sodium ions are detected from the skin by a slight amount even when there is no perspiration. The value that can eliminate the Na value error detected in such a case is used as the constant γ.

### <Threshold Value for Extraction Rate of Sodium Ion J_{Na2}>

As one example of the threshold value for the extraction rate of sodium ion J_{Na2}, the value 0.04 (μmol/h) was used. That is, the case in which the sodium ion amount collected by the perspiration-check purpose collecting material per unit time exceeds 0.04 (μmol/h) is excluded. Using the threshold value, the cases shown in FIG. 13 were divided. The result is shown in FIG. 14.

In FIG. 14, the vertical axis indicates the measurement value deviation rate and the horizontal axis indicates the extraction rate of sodium ion J_{Na2} at the non-puncture site. The measurement value deviation rate is the ratio between the estimated blood glucose AUC and the sampled blood glucose AUC measured for each case. The closer the measurement value deviation rate to 1, the higher the reliability of the estimated blood glucose AUC. As shown in FIG. 14, by setting the threshold value to the extraction rate of sodium ion J_{Na2} to 0.04, many measurement results whose reliability is low, i.e., those results whose measurement value deviation rate is less than 0.8, can be excluded.

### <Threshold Value for Na Relative Value>

As one example of the threshold value for the Na relative value, the value 0.045 was used. Using this threshold value, the cases shown in FIG. 13 were divided. The result is shown in FIG. 15. In FIG. 15, the vertical axis indicates the measurement value deviation rate, and the horizontal axis indicates the Na relative value.

As shown in FIG. 15, by setting the threshold value for the Na relative value to 0.045, many measurement results whose reliability is low, i.e., those results whose measurement value deviation rate is less than 0.8, can be excluded.

When the threshold value for the extraction rate of sodium ion J_{Na2} is used, the case with a great perspiration amount can be excluded without measuring the extraction rate of sodium ion J_{Na1}. Accordingly, wasteful execution of the main measurement can advantageously be prevented for the case where analysis result with low reliability is expected.

When the threshold value for the Na relative value is used, only the case which is relatively largely influenced by the extraction rate of sodium ion J_{Na2} with reference to the extraction rate of sodium ion J_{Na1} can be excluded. Accordingly, as to the case in which the absolute value of the extraction rate of sodium ion J_{Na2} is relatively great but the influence to the main measurement is relatively small, the result of the main measurement can effectively be used instead of being rejected.

Further, in the first embodiment, the mode in which the case with a great perspiration amount is excluded using the threshold value for the extraction rate of sodium ion J_{Na2} is described.

Returning to FIG. 12, when the extraction rate of sodium ion J_{Na2} is equal to or less than the threshold value (“No” in Step S8), in Step S10, the main-measurement purpose collection member 10 is bonded to a prescribed portion of the main-measurement purpose cartridge 40, and the main-measurement purpose cartridge 40 is set to the cartridge disposition portion 22 of the living body component analyzing apparatus 20.

Next, in Step S11, by the living body component analyzing apparatus 20 executing the measurement process, the glucose concentration C_{Glu} and the sodium ion concentration C_{Na1} are measured. Next, the control unit 35 calculates the blood glucose AUC based on the glucose concentration C_{Glu}, the sodium ion concentration C_{Na1}, and the following Formula (1):

AUC=C_{Glu}×V/{α×(C_{Na1}×V/t)+β} (1)

In Formula (1), V is the volume of the collecting material 12 of the main-measurement purpose collection member 10. α and β are each a constant that can empirically be obtained. The principle of calculating the blood glucose AUC based on Formula (1) is detailed in WO 2010/013808 A. WO 2010/013808 A is incorporated herein by reference.

Next, in Step S12, the calculation result is output to the display unit 33 by the control unit 35.

In the present embodiment, sodium ions contained in perspiration from the skin R having not undergone the micropore formation process are collected. By avoiding execution of the main measurement (measurement of C_{Glu} and C_{Na1} and analysis of blood glucose AUC) when the collected sodium ion concentration is higher than the threshold value, blood glucose AUC analysis with low reliability can be prevented.

### [Verification of Effect]

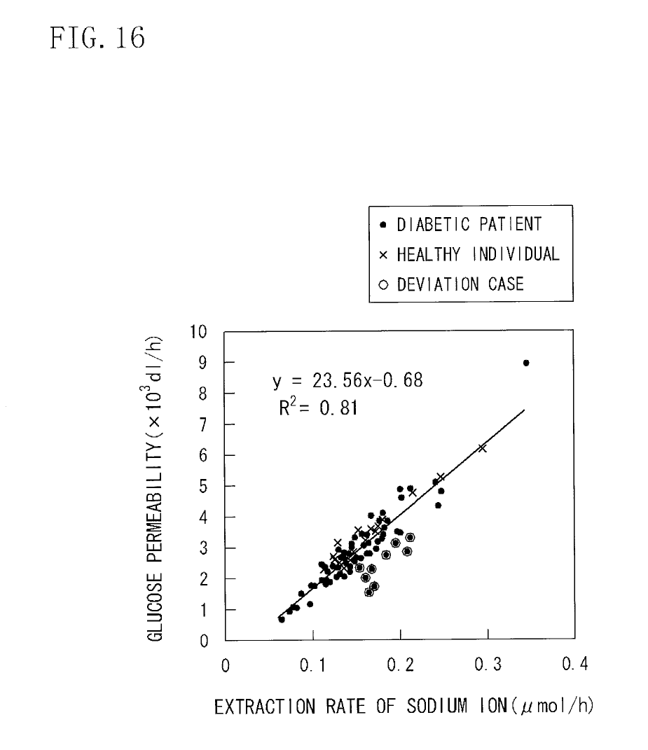

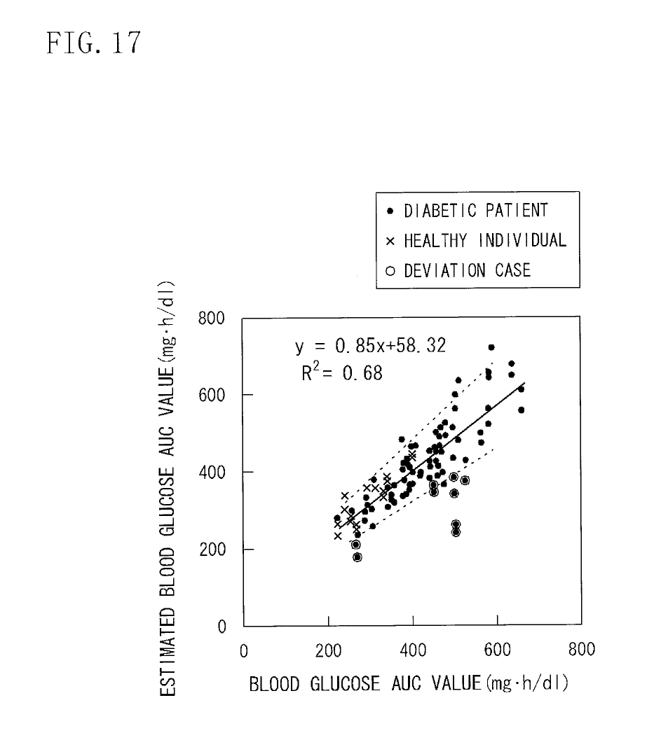

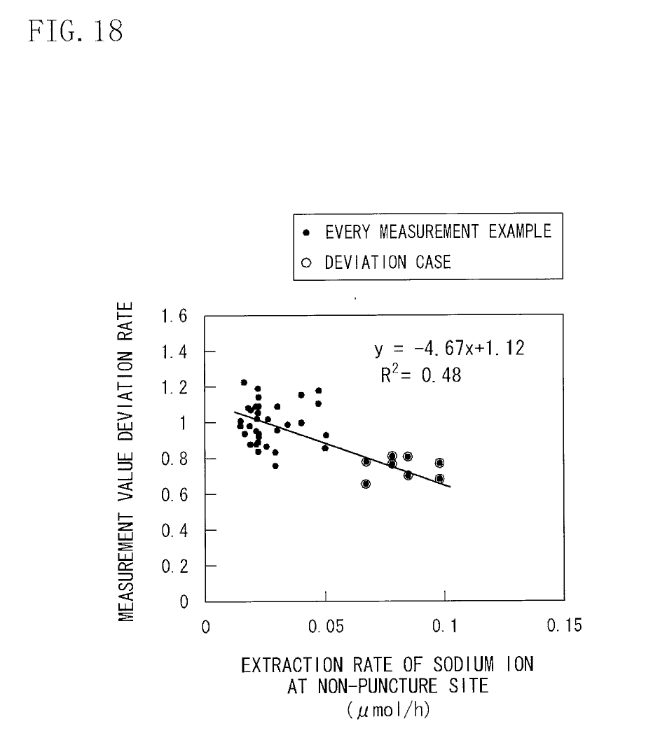

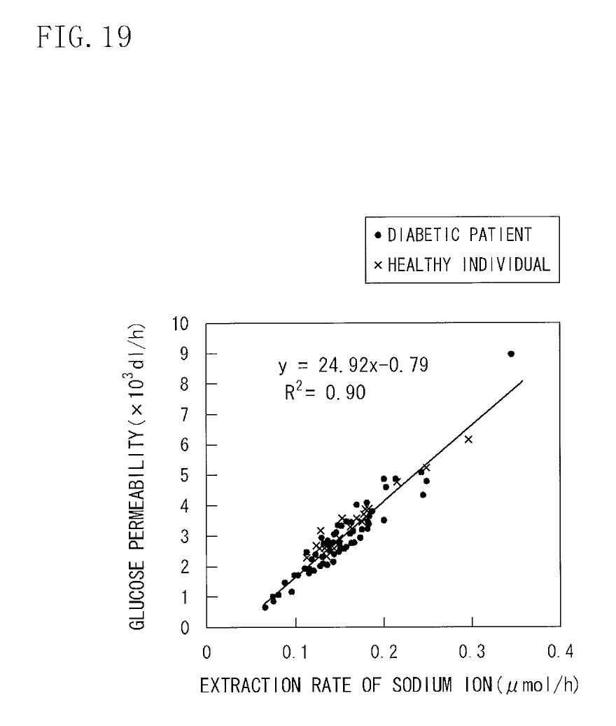

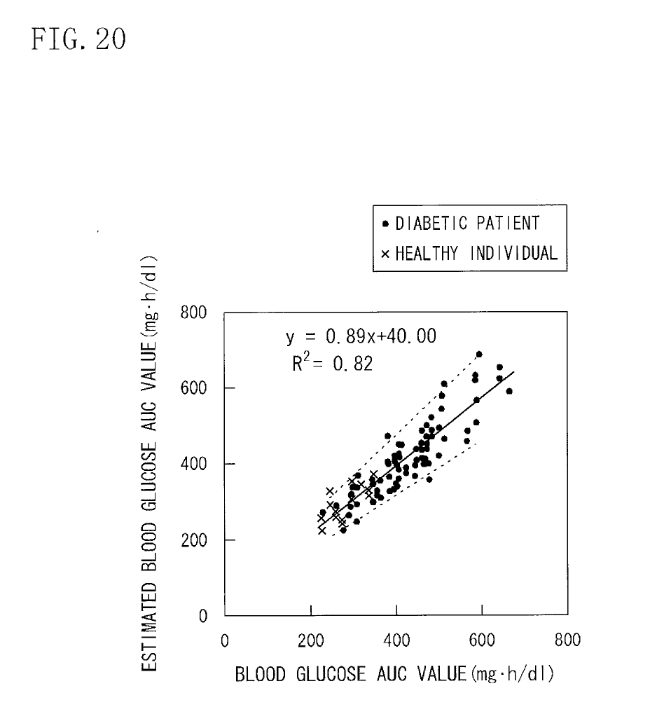

In the following, a description will be given of an example of an improvement in the measurement accuracy of the living body component analyzing method according to the first embodiment. FIG. 16 is a graph showing the correlation between the glucose permeability and the extraction rate of sodium ion before deviation cases are excluded. FIG. 17 is a graph showing the correlation between the estimated blood glucose AUC value and the sampled blood glucose AUC value (the measured blood glucose AUC value). FIG. 18 is a graph showing the relationship between the measurement value deviation rate and the extraction rate of sodium ion at the non-puncture site. FIG. 19 is a graph corresponding to FIG. 16, and is a graph showing the correlation between the glucose permeability and the extraction rate of sodium ion excluding the deviation cases. FIG. 20 is a graph corresponding to FIG. 17, and is a graph showing the correlation between the estimated blood glucose AUC and the sampled blood glucose AUC excluding the deviation cases.

In FIGS. 16 to 18, a symbol “” is the case of a diabetic patient, and a symbol “x” is the case of a healthy individual. Further, the symbol surrounded by ◯ is a deviation case (i.e., a case with a great perspiration amount whose measurement value deviation rate largely deviates from 1). All the deviation cases corresponded to the diabetic patients.

In acquiring the data of FIGS. 16 to 20, in parallel with the collection of the components included in the tissue fluid and perspiration according to the present embodiment, blood sampling was performed for a plurality of times every prescribed time period. The blood glucose AUC was calculated based on the glucose and sodium ions contained in the tissue fluid and the aforementioned Formula (1) (this is referred to as the estimated blood glucose AUC). Further, the blood glucose AUC was calculated by the known trapezoidal approximation from the blood glucose values at a plurality of time points obtained by a plurality of times of blood sampling performed (this is referred to as the sampled blood glucose AUC).

FIG. 16 is a graph in which the vertical axis indicates the glucose permeability and the horizontal axis indicates the extraction rate of sodium ion. The glucose permeability is the value which is the glucose amount collected by the main-measurement purpose collection member 10 being divided by the sampled blood glucose AUC. That is, the glucose permeability represents the ratio of the glucose amount extracted outside the body to the blood glucose AUC inside the body. The extraction rate of sodium ion is the amount of sodium ions collected by the main-measurement purpose collection member 10 per unit time. As detailed in WO 2010/013808 A, the extraction rate of sodium ion and the glucose permeability correlate with each other. As shown in FIG. 16, when the extraction rate of sodium ion and the glucose permeability are plotted, the plot converges around the regression line being a certain gradient.

When the subject perspires, the sodium ions attributed to perspiration are excessively collected by the main-measurement purpose collection member 10. As a result, as represented by circles in FIG. 16, only the extraction rate of sodium ion increases, and the plot shifts to the right side with reference to the regression line. Obtaining the correlation coefficient using every plot point shown in FIG. 16, the correlation coefficient was 0.81.

The correlation between the estimated blood glucose AUC value and the sampled blood glucose AUC value was examined as to the data shown in FIG. 16. The result is shown in FIG. 17.

FIG. 17 is a graph in which the vertical axis indicates the estimated blood glucose AUC and the horizontal axis indicates the sampled blood glucose AUC. As represented by circles in FIG. 17, the cases with great perspiration show great deviation between the estimated blood glucose AUC and the blood glucose AUC, which is attributed to an increase in the sodium ion concentration. Obtaining the correlation coefficient as to every case shown in FIG. 17, the correlation coefficient was 0.68.

FIG. 18 is a graph in which the vertical axis indicates the measurement value deviation rate and the horizontal axis indicates the extraction rate of sodium ion at the non-puncture site. The measurement value deviation rate shown in FIG. 18 is the value which is the estimated blood glucose AUC shown in FIG. 17 being divided by the sampled blood glucose AUC. The closer the measurement value deviation rate to 1, the higher the reliability of the estimated blood glucose AUC. As shown in FIG. 18, the measurement value deviation rate reduces as the extraction rate of sodium ion at the non-puncture site increases. Accordingly, in the present example, the data of five cases and ten sites where the extraction rate of sodium ion at the non-puncture site exceeds 0.06 (μmol/h) is excluded from the analysis target, and analysis is performed again. The result is shown in FIGS. 19 and 20.

When the deviation cases of the five cases and ten sites are excluded from the analysis target, as shown in FIG. 19, the correlation coefficient between the glucose permeability and the extraction rate of sodium ion improved from 0.81 to 0.90. Further, as shown in FIG. 20, the correlation coefficient between the estimated AUC value and the blood glucose AUC value improved from 0.68 to 0.82. Thus, it was demonstrated that avoiding execution of the main measurement as to the case with great perspiration based on the sodium ion amount collected by the perspiration-check purpose collection member 100 prevents the estimated blood glucose AUC value analysis with low reliability from being provided to the user.

### Second Embodiment

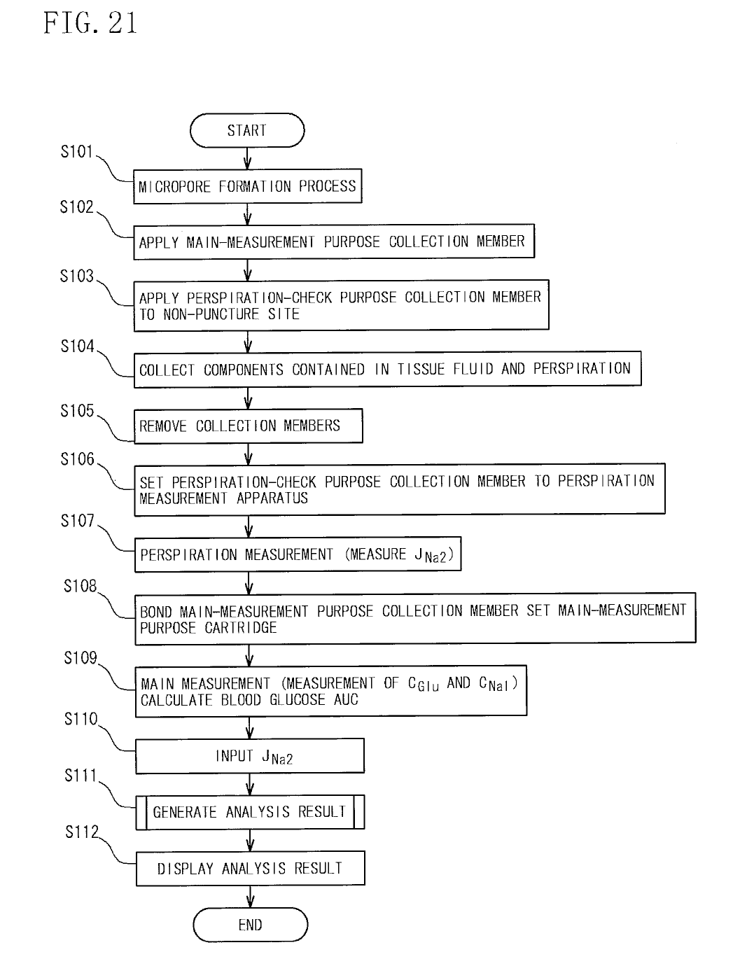

Next, a description will be given of a living body component analyzing method according to a second embodiment of the present invention. FIG. 21 is a flowchart of the living body component analyzing method according to the second embodiment.

What has exemplarily been shown in the first embodiment is the mode in which whether or not main measurement (measurement of C_{Glu} and C_{Na1}) and calculation of blood glucose AUC is to be executed is determined based on the perspiration measurement result (J_{Na2}). In the second embodiment, the perspiration measurement and the main measurement are previously executed, and information on the reliability of the blood glucose AUC is output together with the analysis result of the blood glucose AUC.

In the flowchart of FIG. 21, the steps of Steps S101 to 105 are the same as Steps S1 to 5 according to the first embodiment shown in FIG. 12 and, therefore, detailed description of Steps S101 to S105 is not given herein, and the analyzing steps of Steps S106 to S111 are detailed. Steps S106 to S111 are the steps of analyzing the components collected in Step S104.

First, in Step S106, the perspiration-check purpose collection member 100 is set to the perspiration measurement apparatus 60. In Step S107, the concentration C_{Na2} of sodium ions collected by the collecting material 120 is measured. Next, in Step S108, the main-measurement purpose collection member 10 is bonded to a prescribed portion of the main-measurement purpose cartridge 40, and the main-measurement purpose cartridge 40 is set to the cartridge disposition portion 22 of the living body component analyzing apparatus 20. In Step S109, the glucose concentration C_{Glu} and the sodium ion concentration C_{Na1} collected by the collecting material 12 are measured. Based on the glucose concentration C_{Glu} and the sodium ion concentration C_{Na1}, the estimated blood glucose AUC value is calculated.

Next, in Step S110, the extraction rate of sodium ion J_{Na2} obtained in Step S107 is input to the living body component analyzing apparatus 20 by the user. In Step S111, the analysis result is generated by the control unit 35, and the generated analysis result is output to the display unit 33 in Step S112.

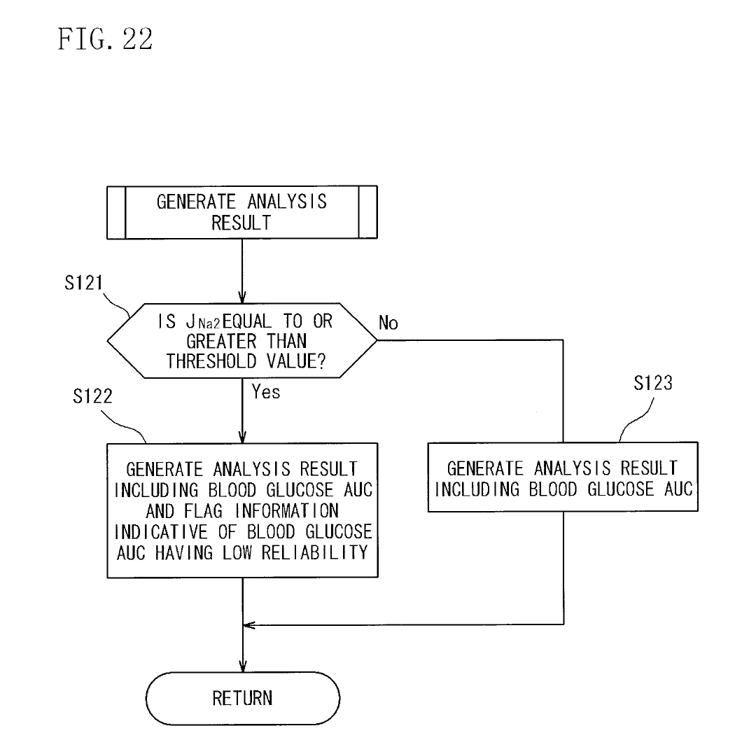

FIG. 22 is a flowchart of the process executed by the control unit 35 in Step S111.

First, in Step S121, the control unit 35 compares the input extraction rate of sodium ion J_{Na2} with threshold value, to determine whether or not the extraction rate of sodium ion J_{Na2} is equal to or greater than the threshold value. When the control unit 35 determines that it is equal to or greater than the threshold value (YES in Step S121), the control unit 35 proceeds to Step S122. In Step S122, the control unit 35 generates an analysis result that includes the blood glucose AUC calculated in Step S109 and flag information indicative of the blood glucose AUC having low reliability. On the other hand, when the control unit 35 determines that the extraction rate of sodium ion J_{Na2} is less than the threshold value (NO in Step S122), the control unit 35 proceeds to Step S123. In Step S123, the control unit 35 generates an analysis result that includes only the blood glucose AUC calculated in Step S109. When the extraction rate of sodium ion J_{Na2} is less than the threshold value, the blood glucose AUC has high reliability. Therefore, flag information is not included in the analysis result.

According to the second embodiment, when the subject perspires to the extent that it may influence the calculation result of the blood glucose AUC, the user can be notified that the reliability of the blood glucose AUC is reduced. The user can use this flag information in making a determination as to whether or not to use the output blood glucose AUC.

It is to be noted that, though the mode in which J_{Na2} is compared with the threshold value has been shown in the second embodiment, it is also possible to employ the mode in which the Na relative value is obtained based on J_{Na1} and J_{Na2}, and the Na relative value and the threshold value are compared with each other.

### Third Embodiment

In the first and second embodiments described above, one threshold value is set, and the determination as to whether or not the extraction rate of sodium ion J_{Na2} is higher than the threshold value is made. However, it is also possible to set a plurality of stepwise threshold values. For example, it is also possible to set two threshold values (the first threshold value and the second threshold value greater than the first threshold value), and to generate different analysis results depending on the extraction rate of sodium ion J_{Na2}.

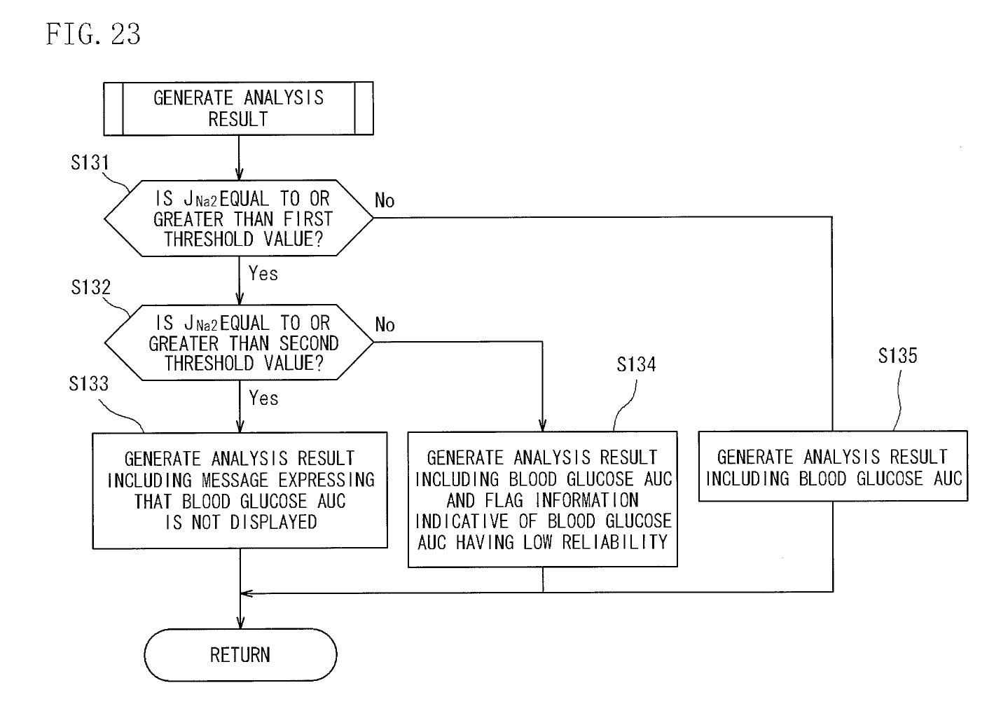

FIG. 23 is a flowchart showing processing of a control unit in a living body component analyzing method according to a third embodiment. Since the third embodiment is identical to Steps S101 to S112 shown in FIG. 21 except for the processing by the control unit 35, a description thereof will not be repeated herein.

First, in Step S131, the control unit 35 determines as to whether or not the extraction rate of sodium ion J_{Na2} is equal to or greater than the first threshold value. Here, as the first threshold value, the value that does not necessitate rejection of the analysis result of the blood glucose AUC but that influences the analysis of the blood glucose AUC is set. When the control unit 35 determines that the extraction rate of sodium ion J_{Na2} is equal to or greater than the first threshold value (YES in Step S131), the control unit 35 proceeds to Step S132. When the control unit 35 determines that the sodium ion concentration C_{Na2} is less than the first threshold value (NO in Step S131), the control unit 35 proceeds to Step S136.

In Step S136, the control unit 35 generates the analysis result that includes only the blood glucose AUC calculated in Step S109.

In Step S132, the control unit 35 determines as to whether or not the extraction rate of sodium ion J_{Na2} is equal to or greater than the second threshold value. Here, as the second threshold value, the value that is greater than the first threshold value and that necessitates rejection of the analysis result of the blood glucose AUC is set. When the control unit 35 determines that the extraction rate of sodium ion J_{Na2} is equal to or greater than the second threshold value (YES in Step S132), the control unit 35 proceeds to Step S133. When the control unit 35 determines that the extraction rate of sodium ion J_{Na2} is less than the second threshold value (NO in Step S132), the control unit 35 proceeds to Step S134.

In Step S134, the control unit 35 generates an analysis result that includes the blood glucose AUC calculated in Step S109 and the flag information to the effect that the blood glucose AUC has low reliability.

In Step S133, the control unit 35 generates an analysis result that includes a message expressing “The analysis result of the blood glucose AUC cannot be displayed because the reliability of the analysis result cannot be guaranteed. Please perform measurement again”. In this case, the blood glucose AUC calculated in Step S109 is not included in the analysis result.

According to the third embodiment, the analysis result that differs depending on the perspiration amount of the subject can be output. Further, when the subject perspires to the extent that necessitates rejection of the analysis result of the blood glucose AUC, by outputting the message expressing that the analysis result of the blood glucose AUC is not to be output, it becomes possible to prompt the user to perform measurement again when the perspiration amount is suppressed.

It is to be noted that, though the mode in which J_{Na2} is compared with the threshold value has been shown in the third embodiment, it is also possible to employ the mode in which the Na relative value is obtained based on J_{Na1} and J_{Na2}, and the Na relative value and the threshold value are compared with each other.

### Fourth Embodiment

In the embodiments described above, J_{Na2} is used as the criterion in making a determination as to whether or not the main measurement is to be started (the first embodiment); or the main measurement is performed subsequent to the measurement of J_{Na2}, and depending on the value of J_{Na2}, the display to the effect that the reliability of the analysis result of the blood glucose AUC is low is presented (the second or third embodiment). However, it is also possible to improve the accuracy of the measurement target component analysis by performing correction processing, such as by subtracting the value relating to the amount of the second auxiliary component from the value relating to the amount of the first auxiliary component.

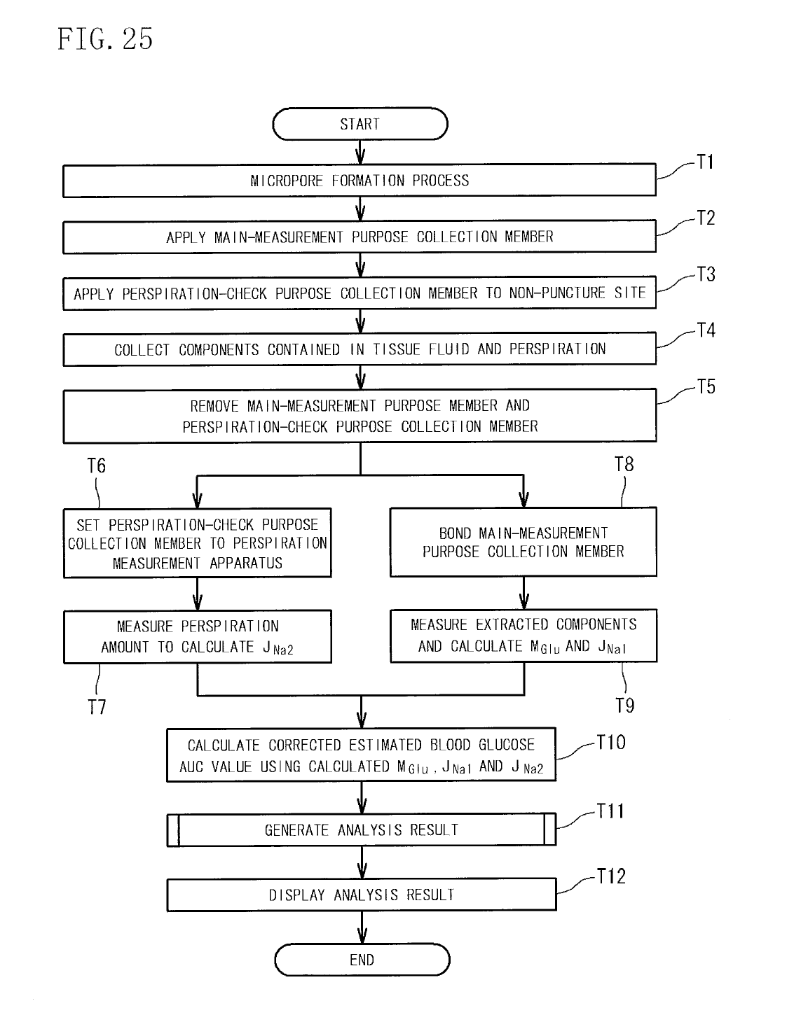

In the present embodiment, the accuracy of the analysis of the measurement target component is improved by performing such correction processing. FIG. 25 is a flowchart of a living body component analyzing method according to a fourth embodiment.

The processing from Step T1 (the micropore formation process) to Step T5 (removal of the main-measurement purpose collection member and the perspiration-purpose collection member) is the same as that of Steps S1 to S5 according to the first embodiment shown in FIG. 12. Therefore, for sake of simplicity, the description thereof is not repeated.

Steps T6 to T10 are the steps of analyzing the components collected in Step T4. In the present embodiment, in order to reduce the analysis time, the components collected by the perspiration-check purpose collection member and the components collected by the main-measurement purpose collection member are analyzed in parallel to each other.

First, in Step T6, the perspiration-check purpose collection member 100 removed from the skin of the subject is set to the perspiration measurement apparatus 60. The perspiration-check purpose collection member 100 is set to the perspiration measurement apparatus 60 such that the opposing electrodes 61a and 61b of the perspiration measurement apparatus 60 are buried in the collecting material 120 of the perspiration-check purpose collection member 100.

Next, in Step T7, by measuring the conductivity of the collecting material 120 of the perspiration-check purpose collection member 100, the sodium ion concentration C_{Na2} contained in the collecting material 120 is measured. The sodium ion concentration C_{Na2} measured by the perspiration measurement apparatus 60 is displayed on the display unit 60c. Then, when the measured sodium ion concentration C_{Na2} is input to the living body component analyzing apparatus 20 by the manipulation button 34, the control unit 35 calculates the extraction rate of sodium ion J_{Na2} of the non-puncture site based on the input sodium ion concentration C_{Na2} and according to the following formula:

J_{Na2}=C_{Na2}×V₂/t

Here, V₂ is the volume of the collecting material 120 of the perspiration-check purpose collection member 100, and t is the extraction time.

On the other hand, in parallel with Step T6, in Step T8, the main-measurement purpose collection member 10 is bonded to a prescribed portion of the main-measurement purpose cartridge 40, and the main-measurement purpose cartridge 40 is set to the cartridge disposition portion 22 of the living body component analyzing apparatus 20.

Next, in Step T9, by the living body component analyzing apparatus 20 executing the measurement process described above, the glucose concentration C_{Glu} and the sodium ion concentration C_{Na1} are measured. Next, the control unit 35 calculates the extracted glucose amount M_{Glu} and the extraction rate of sodium ion J_{Na1} at the puncture site, based on the glucose concentration C_{Glu}, the sodium ion concentration C_{Na1}, and the following formulas:

M_{Glu}=C_{Glu}×V₁

J_{Na1}=C_{Na1}×V₁/t

Here, V₁ is the volume of the collecting material 12 of the main-measurement purpose collection member 10, and t is the extraction time.

Next, in Step T10, the control unit 35 calculates the corrected estimated blood glucose AUC value using J_{Na2} calculated in Step T7 and M_{Glu} and J_{Na1} calculated in Step T9 and according to the following Formula (2):

$$AUC = \frac{{M}_{Glu}}{α \times ( {J}_{Na 1} - {J}_{Na 2} ) + β} \qquad ( 2 )$$

Here, α and β are each a constant that is empirically obtained.

Next, in Step T11, the analysis result is generated by the control unit 35, and the generated analysis result is output to the display unit 33 in Step T12.

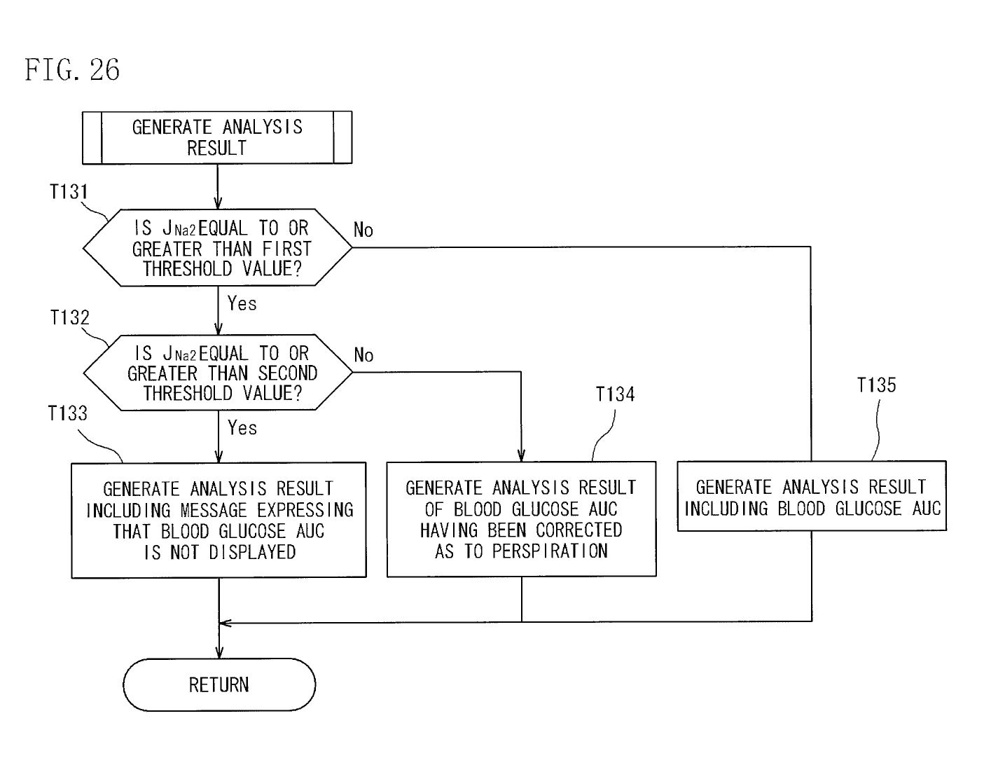

FIG. 26 is a flowchart showing the processing of the control unit in the living body component analyzing method according to the fourth embodiment.

First, in Step T131, the control unit 35 determines whether or not the extraction rate of sodium ion J_{Na2} is equal to or greater than the first threshold value. Here, as the first threshold value, the value that does not necessitate rejection of the blood glucose AUC analysis result but that influences the analysis of the blood glucose AUC is set. When the control unit 35 determines that the extraction rate of sodium ion J_{Na2} is equal to or greater than the first threshold value (Yes in Step T131), the control unit 35 proceeds to Step T132. When the control unit 35 determines that the extraction rate of sodium ion J_{Na2} is less than the first threshold value (No in Step T131), the control unit 35 proceeds to Step T136.

In Step T136, the control unit 35 generates the analysis result that includes the blood glucose AUC calculated in Step T9.

In Step T132, the control unit 35 determines as to whether or not the extraction rate of sodium ion J_{Na2} is equal to or greater than the second threshold value. Here, as the second threshold value, the value that is greater than the first threshold value and that necessitates rejection of the analysis result of the blood glucose AUC is set. When the control unit 35 determines that the extraction rate of sodium ion J_{Na2} is equal to or greater than the second threshold value (Yes in Step T132), the control unit 35 proceeds to Step T133. When the control unit 35 determines that the extraction rate of sodium ion J_{Na2} is less than the second threshold value (No in Step T132), the control unit 35 proceeds to Step T134.

In Step T134, the control unit 35 generates the analysis result that includes the corrected estimated blood glucose AUC value calculated in Step T10.

In Step T133, the control unit 35 generates the analysis result that includes a message expressing “The analysis result of the blood glucose AUC cannot be displayed because the reliability of the analysis result cannot be guaranteed. Please perform measurement again”. In this case, the blood glucose AUC calculated in Steps T9 and T10 is not included in the analysis result.

According to the fourth embodiment, the analysis result that differs depending on the perspiration amount of the subject can be output. Further, employing such a structure, when the subject perspires to the extent that the influence of the perspiration can be corrected, the estimated blood glucose AUC value can be output using the correction value. Further, when the subject so perspires that rejection of the analysis result of the blood glucose AUC becomes necessary, by outputting the message to the effect that the analysis result of the blood glucose AUC is not to be output, it becomes possible to prompt the user to perform measurement again when the perspiration amount is suppressed.

### [Verification of Effect]

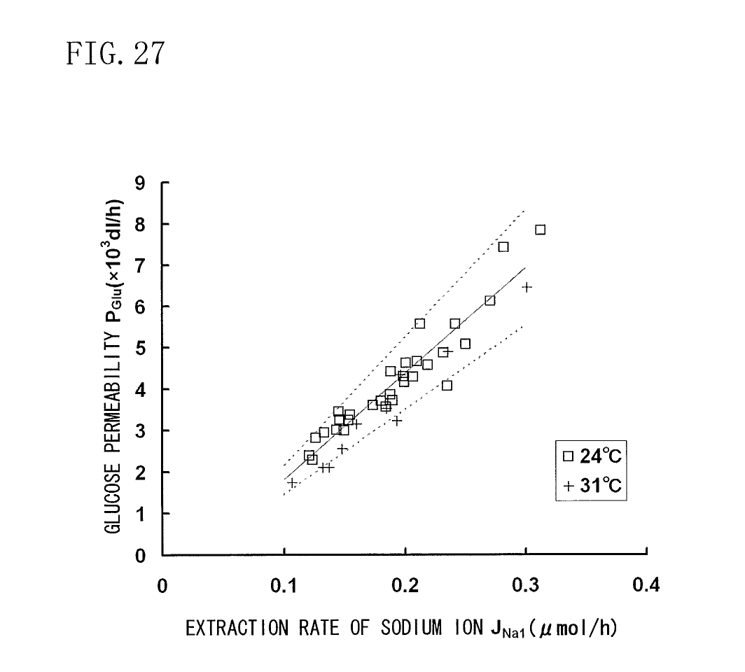

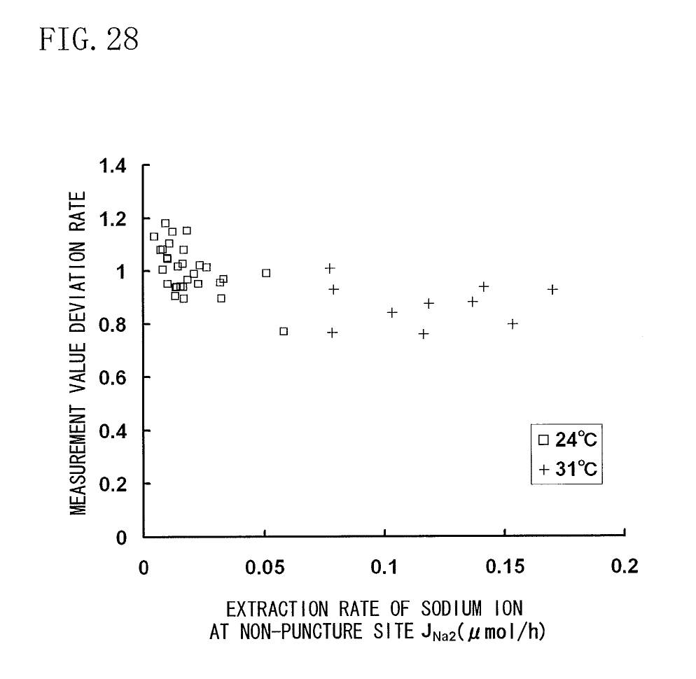

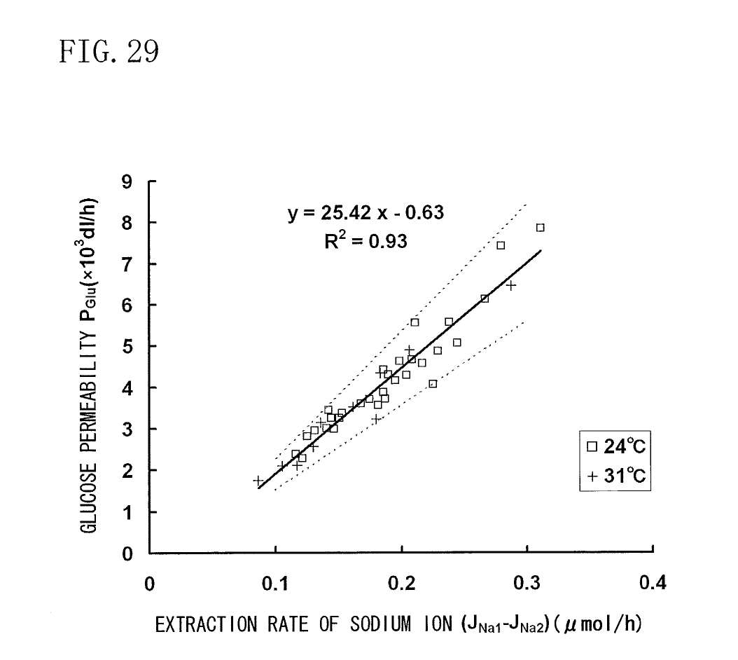

In the following, a description will be given of an example of an improvement in the measurement accuracy through the living body component analyzing method according to the fourth embodiment. FIG. 27 is a graph showing the correlation between the glucose permeability (P_{Glu}) and the extraction rate of sodium ion (J_{Na1}) as to a plurality of subjects when the perspiration correction described above is not performed. FIG. 28 is a graph showing the relationship between the extraction rate of sodium ion (J_{Na2}) and the measurement value deviation rate at the non-puncture site. FIG. 29 is a graph showing the correlation between the glucose permeability (P_{Glu}) and the extraction rate of sodium ion (J_{Na1}-J_{Na2}) when the perspiration correction is performed by the aforementioned Formula (2). In FIGS. 27 to 29 and FIG. 30 whose description will follow, a symbol “□” represents an experimental example under the condition of room temperature being 24° C., while a symbol “+” represents an experimental example under the condition of room temperature being 31° C.

FIG. 27 is a graph in which the vertical axis indicates the glucose permeability, and the horizontal axis indicates the extraction rate of sodium ion. The glucose permeability is the value which is the glucose amount collected by the main-measurement purpose collection member 10 being divided by the sampled blood glucose AUC. That is, the glucose permeability represents the ratio of the glucose amount extracted outside the body to the blood glucose AUC inside the body. The extraction rate of sodium ion is the amount of the sodium ions collected by the main-measurement purpose collection member 100 per unit time. As detailed in WO 2010/013808 A, the extraction rate of sodium ion and the glucose permeability correlate with each other. As shown in FIG. 27, when the extraction rate of sodium ion and the glucose permeability are plotted, the plot converges around the regression line being a certain gradient.

However, when the subject perspires, the sodium ions attributed to perspiration are excessively collected by the main-measurement purpose collection member 10. This tendency increases as the room temperature is higher. With the measurement at 31° C., since the sodium ion amount attributed to perspiration is further added, the distribution deviates in the right direction with reference to the measurement data at 24° C. with small perspiration. Obtaining the correlation coefficient using every plot shown in FIG. 27, the correlation coefficient was 0.95. Further, the measurement error (the standard deviation of the measurement value deviation rate) was 10.8%. Further, three cases out of ten cases subjected to the measurement at 31° C. showed the low values being equal to or less than the measurement value deviation rate 0.8 because of perspiration.

From FIG. 28, it can be seen that, under the condition of 31° C., the extraction speed of the sodium ions attributed to perspiration largely increases as compared to the case under the condition of 24° C., and the cases where the measurement value deviation rate is equal to or less than 0.8 increases.

In contrast thereto, performing the perspiration correction of subtracting the extraction rate of sodium ion J_{Na2} at the non-puncture site from the extraction rate of sodium ion J_{Na1} at the puncture site, as shown in FIG. 29, the distribution bias of the measurement data of 24° C. and 31° C. was solved, and the measurement accuracy was improved. Obtaining the correlation coefficient using every plot shown in FIG. 29, the correlation coefficient was 0.96. Further, the measurement error was reduced to 8.6%. Still further, there was no case whose measurement deviation rate is less than 0.8.

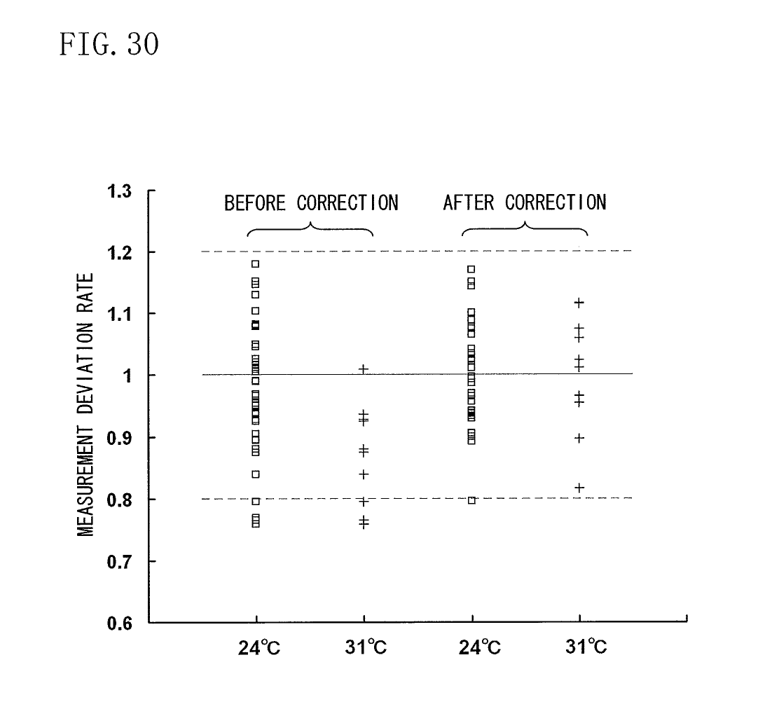

FIG. 30 is a graph showing the measurement value deviation rate before and after correction. There was no great change at 24° C. with small perspiration. At 31° C. with great perspiration, though the average value of the measurement value deviation rate before correction was approximately 0.87, the average value after correction was approximately 1.0. Thus, an improvement in measurement accuracy was demonstrated.

### [Other Variation]

It is to be noted that the present invention is not limited to the embodiments described above, and various changes can be made.

In the embodiments described above, the examples where glucose is measured as the measurement target component have been shown. However, the present invention is not limited thereto, and the amount of substance other than glucose included in the tissue fluid may be measured. The substance measured by the present invention may be, for example, biochemical components or any drug administered to the subject. The biochemical components may include protein being one type of biochemical components, i.e., albumin, globulin, enzyme and the like. Further, the biochemical components other than protein may include creatinine, creatine, uric acid, amino acid, fructose, galactose, pentose, glycogen, lactic acid, pyruvic acid, ketone body and the like. Still further, the drug agent may include digitalis preparation, theophylline, antiarrhythmic agents, antiepileptic agents, amino acid sugar antibiotics, glycopeptide antibiotics, antithrombotic agents, immunosuppressive agents and the like.

Further, in the embodiments described above, the examples where sodium ions are used as the first auxiliary component and the second auxiliary component have been shown. However, the present invention is not limited thereto.

For example, the first auxiliary component and the second auxiliary component may be the components being different from each other.

The first auxiliary component is only required to be the component that is contained in the body at a certain concentration and that reflects the micropore formation state, and inorganic ions such as potassium ions, calcium ions, magnesium ions or the like can be used in place of the sodium ions.

The second auxiliary component is only required to be the component that is contained in perspiration, and it may be inorganic ions such as potassium ions, calcium ions, magnesium ions or moisture or the like can be used in place of the sodium ions.

Still further, in the embodiments described above, the examples where the perspiration measurement apparatus 60 and the living body component analyzing apparatus 20 performing the main measurement are separate apparatuses have been shown. However, these apparatuses can be integrated. In this case, separately from the main-measurement purpose cartridge 40 to which the main-measurement purpose collection member 10 is bonded, a perspiration-check purpose cartridge for bonding the perspiration-check purpose collection member 100 may be prepared. By disposing the perspiration-check purpose cartridge in the disposition portion 22 of the living body component analyzing apparatus 20, the perspiration measurement and the main measurement may be performed in the living body component analyzing apparatus 20. As still another variation, a common cartridge to which both the main-measurement purpose collection member 10 and the perspiration-check purpose collection member 100 can be bonded is prepared, and an analyzing apparatus that can accommodate this cartridge may be provided. Thus, the components collected by the two collection members can simultaneously be analyzed.

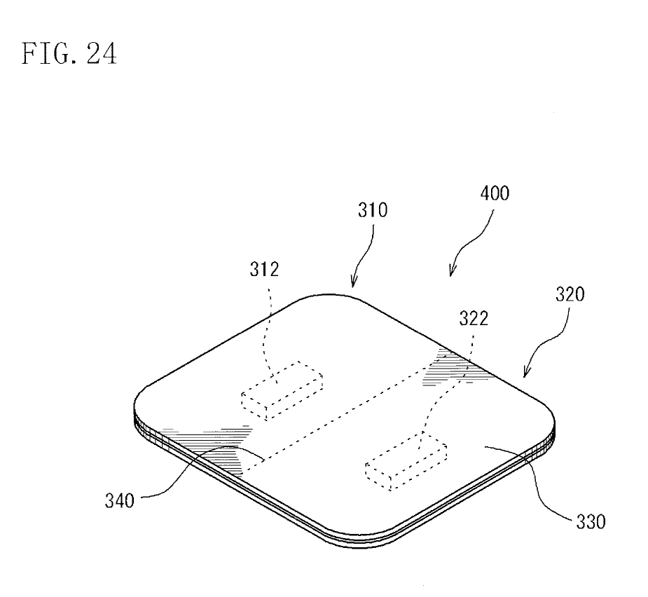

Still further, in the embodiments described above, the examples where the main-measurement purpose collection member 10 and the perspiration-check purpose collection member 100 are prepared as separate members have been shown. However, as shown in FIG. 24, the two members may be integrated. FIG. 24 is a perspective view showing the structure of an integrated collection member 400. The integrated collection member 400 includes a main-measurement purpose first collecting unit 310 and a perspiration-check purpose second collecting unit 320. The first collecting unit 310 is similarly structured as the main-measurement purpose collection member 10 described above, and includes a main-measurement purpose collecting material 312. The second collecting unit 320 is similarly structured as the perspiration-check purpose collection member 100 described above, and includes a perspiration-check purpose collecting material 322. The collecting material 312 and the collecting material 322 are retained by an integrated retainer sheet 330. The integrated retainer sheet 330 is similarly structured as the retainer sheet described above. The integrated retainer sheet 330 is provided with perforation 340 for separating the first collecting unit 310 and the second collecting unit 320 from each other.

The integrated collection member 400 is used as follows. The integrated retainer sheet 330 is applied to the skin of the subject such that the main-measurement purpose collecting material 312 is positioned at the micropore formation region S and the perspiration-check purpose collecting material 322 is positioned at the non-puncture site R. After the collection of the components has finished, the integrated collection member 400 is removed from the skin, and the first collecting unit 310 and the second collecting unit 320 are separated from each other along the perforation 340. The first collecting unit 310 undergoes the main measurement by the living body component analyzing apparatus 20, and the second collecting unit 320 undergoes the perspiration measurement by the perspiration measurement apparatus 60.

In this manner, by integrating the collection members, perspiration can be collected at the position near the site where the tissue fluid is extracted (the puncture site S). Hence, the difference in the collected perspiration amount between the first collecting unit 310 and the second collecting unit 320 can be reduced.

Further, in the embodiments described above, the examples where the extraction rate of sodium ion J_{Na2} measured by the perspiration measurement apparatus is input by the user to the living body component analyzing apparatus have been described. However, the present invention is not limited thereto. For example, it is also possible to connect the perspiration measurement apparatus and the living body component analyzing apparatus so as to be capable of establishing communication with each other, and to transmit the result (data) obtained by the perspiration measurement apparatus to the control unit of the living body component analyzing apparatus.

What is claimed is:

1. A living body component analyzing method for analyzing a component contained in a tissue fluid extracted from a skin of a subject, including:
a step of subjecting part of a skin of a subject to a process of facilitating extraction of a tissue fluid;
a step of collecting a measurement target component from the skin subjected to the facilitation process;
a step of collecting a first auxiliary component from the skin subjected to the facilitation process;
a step of collecting a second auxiliary component contained in perspiration from the skin excluding the part of the skin subjected to the facilitation process; and
a step of analyzing the measurement target component based on the collected measurement target component, the collected first auxiliary component, and the collected second auxiliary component.

2. The living body component analyzing method according to claim 1, wherein
the first auxiliary component and the second auxiliary component are collected in an identical period.

3. The living body component analyzing method according to claim 1, wherein
the first auxiliary component and the second auxiliary component are collected at an identical arm.

4. The living body component analyzing method according to claim 1, wherein
the step of analyzing includes:
a first measurement step of measuring the collected second auxiliary component to acquire a first measurement value;
a step of comparing the first measurement value with a prescribed threshold value;
a second measurement step of measuring the collected measurement target component to acquire a second measurement value, when the first measurement value is smaller than the prescribed threshold value;
a third measurement step of measuring the collected first auxiliary component to acquire a third measurement value, when the first measurement value is smaller than the prescribed threshold value; and
a step of generating an analysis result including a value relating to an amount of the measurement target component based on the second and third measurement values.

5. The living body component analyzing method according to claim 1, wherein
the step of analyzing includes:
a first measurement step of measuring the collected second auxiliary component to acquire a first measurement value;
a second measurement step of measuring the collected measurement target component to acquire a second measurement value;
a third measurement step of measuring the collected first auxiliary component to acquire a third measurement value; and
a step of generating an analysis result of the measurement target component based on the first to third measurement values.

6. The living body component analyzing method according to claim 5, wherein
the step of generating the analysis result includes:
a step of comparing the first measurement value with the prescribed threshold value; and
a step of generating an analysis result including a value relating to an amount of the measurement target component based on the second and third measurement values and information indicative of the value having low reliability, when the first measurement value is equal to or greater than the prescribed threshold value.

7. The living body component analyzing method according to claim 5, wherein
the step of generating the analysis result includes:
a step of comparing the first measurement value with the prescribed threshold value; and
a step of generating an analysis result including a message expressing that the value relating to the amount of the measurement target component is not to be output, when the first measurement value is equal to or greater than the prescribed threshold value.

8. The living body component analyzing method according to claim 5, wherein
the step of generating the analysis result includes:
a step of comparing the first measurement value with the first threshold value and with a second threshold value, the second threshold value being greater than the first threshold value;
a step of generating an analysis result including a value relating to an amount of the measurement target component based on the second and third measurement values and information indicative of the value having low reliability, when the first measurement value is equal to or greater than the first threshold value and smaller than the second threshold value; and
a step of generating an analysis result including a message expressing that the value relating to the amount of the measurement target component is not to be output, when the first measurement value is equal to or greater than the second threshold value.

9. The living body component analyzing method according to claim 5, wherein
the first measurement value is a value relating to an amount of the second auxiliary component;
the second measurement value is a value relating to an amount of the measurement target component; and
the third measurement value is a value relating to an amount of the first auxiliary component.

10. The living body component analyzing method according to claim 9, wherein
the step of analyzing is a step of generating an analysis result of the measurement target component by correcting the second measurement value by a correction value obtained based on the first measurement value and the third measurement value.

11. The living body component analyzing method according to claim 10, wherein
the correction value is a value obtained by subtracting the first measurement value from the third measurement value.

12. The living body component analyzing method according to claim 11, wherein
each of the values relating to the amount is an extraction amount of each of the auxiliary components per unit time.

13. The living body component analyzing method according to claim 9, wherein
the step of generating the analysis result includes:
a step of comparing the first measurement value with the first threshold value and with a second threshold value, the second threshold value being greater than the first threshold value;
a step of generating an analysis result of the measurement target component by correcting the second measurement value by a correction value based on the first and third measurement values, when the first measurement value is equal to or greater than the first threshold value and smaller than the second threshold value; and
a step of generating an analysis result including a message expressing that the value relating to the amount of the measurement target component is not to be output, when the first measurement value is equal to or greater than the second threshold value.

14. The living body component analyzing method according to claim 1, wherein
the measurement target component is glucose.

15. The living body component analyzing method according to claim 1, wherein
the first auxiliary component and the second auxiliary component are inorganic ions.

16. The living body component analyzing method according to claim 1, wherein
the first auxiliary component and the second auxiliary component are of an identical type.

17. The living body component analyzing method according to claim 15, wherein
the inorganic ions are sodium ions.

18. The living body component analyzing method according to claim 1, wherein
the measurement target component and the first auxiliary component are collected by a collecting material disposed at an application face of a retainer sheet, the application face being capable of being applied to the skin of the subject.

19. The living body component analyzing method according to claim 18, wherein
the collecting material is made of gel.

20. A living body component analyzing apparatus for analyzing a component contained in a tissue fluid extracted from a skin of a subject, comprising:
an acquiring unit that acquires information relating to a measurement target component and a first auxiliary component from a collection member having been disposed for a prescribed time at part of the skin of the subject, the part being subjected to a process of facilitating extraction of the component; and
an analyzing unit that analyzes the measurement target component based on the information relating to the measurement target component and to the first auxiliary component acquired by the acquiring unit, and based on information relating to a second auxiliary component contained in perspiration from the skin excluding the part of the skin subjected to the facilitation process.

21. The living body component analyzing apparatus according to claim 20, further comprising:
a second acquiring unit that acquires information relating to the second auxiliary component.

22. The living body component analyzing apparatus according to claim 20, further comprising:
an information receiving unit that receives the information relating to the second auxiliary component.
